(19) United States
(12) Patent Application Publication
(10) Pub. No.: US 2007/0043070 A1
(43) Pub. Date: Feb. 22, 2007

(54) Weak base salts

(76) Inventors: Samuel H. Yalkowsky, Tucson, AZ (US); Tapan Sanghvi, Tucson, AZ (US)

Correspondence Address: GREENLEE WINNER AND SULLIVAN P C, 4875 PEARL EAST CIRCLE, SUITE 200, BOULDER, CO, 80301, US

(21) Appl. No.: 10/548,000

(22) PCT Filed: Mar. 12, 2004
(86) PCT No.: PCT/US04/07786

§ 371 Date: Sep. 19, 2006

(30) Foreign Application Priority Data
Nov. 1, 2000 (US) 09/703,955

(51) Int. Cl. C07D 471/02; A61K 31/4745

(52) U.S. Cl. 514/303; 546/117

(57) ABSTRACT

Pharmaceutical compositions comprising a salt of a weak base compound of formula: wherein X is hydrogen, halogen, alkyl of less than 7 carbon atoms or alkoxy of less than 7 carbon atoms; n is a positive integer of less than 4; Y is hydrogen, chlorine, nitro, methyl, ethyl or oxy-chloro; R is hydrogen, alkylaminocarbonyl wherein the alkyl group has from 3 to 6 carbon atoms or an alkyl group having from 1 to 8 carbons and R2 is 4-thiazolyl, NHCOOR1 wherein R, is aliphatic hydrocarbon of less than 7 carbon atoms, or an alkyl group of less than 7 carbon atoms; one or more free acids; and optional pharmaceutical additives are provided.

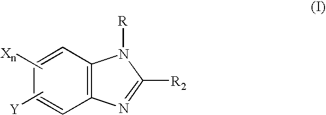

### ACKNOWLEDGMENT OF FEDERAL RESEARCH SUPPORT

This invention was made, at least in part, with funding from the National Institutes of Health. Accordingly, the United States Government may have certain rights to this invention.

### BACKGROUND OF THE INVENTION

Most active pharmaceutical ingredients are poorly soluble in water, and therefore provide a challenge to formulators in developing a therapeutically viable formulation. A number of solubilization techniques that involve modification of either the solute or the solvent have been described to overcome this challenge. If a compound possesses an ionization centre, then the possibility of forming a salt exists. Salt formation provides a means of altering the physicochemical and resultant biological characteristics of a drug without modifying its chemical structure. Factors that can be changed by salt formation include solubility, dissolution, hygroscopicity, taste, physical and chemical stability or polymorphism. Water soluble salts allow the preparation of injectable sterile aqueous solutions, and rapid dissolution of the active component contained in solid dosage form.

This invention is in the field of improving the water solubility of benzimidazole derivatives and other weak bases and providing pharmaceutical formulations of the same. Benzimidazole derivatives are useful for inhibiting the growth of cancers, tumors and viruses in mammals, particularly in humans and warm-blooded animals (U.S. Pat. Nos. 6,479,526; 5,880,144; 6,245,789; 5,767,138; 6,265,437). Certain benzimidazole derivatives used in combination with other compounds have been reported to be useful as fungicides (U.S. Pat. Nos. 3,954,993; 4,593,040; 5,756,500; 4,835,169; 4,980,346). However, benzimidazole derivatives, including carbendazim, are poorly water soluble. The projected oral dose of carbendazim for cancer treatment is up to several hundred mg per day which is far greater than its water solubility. Other weak bases suffer from the same poor water solubility.

There is a need for improved formulations of benzimidazole derivatives and other weak bases.

### BRIEF SUMMARY OF THE INVENTION

Provided are salts of weak bases having formula: 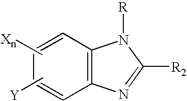
wherein X is hydrogen, halogen, alkyl of less than 7 carbon atoms or alkoxy of less than 7 carbon atoms; n is a positive integer of less than 4; Y is hydrogen, chlorine, nitro, methyl, ethyl or oxychloro; R is hydrogen, alkylaminocarbonyl wherein the alkyl group has from 3 to 6 carbon atoms or an alkyl group having from 1 to 8 carbons, and R₂ is 4-thiazolyl, NHCOOR₁ wherein R₁ is an aliphatic hydrocarbon of less than 7 carbon atoms, or an alkyl group of less than 7 carbon atoms. The salt is preferably one or more selected from the group consisting of: chlorides, bromides, phosphates, sulfates, tosylates, benzoylates, nitrates, sulfonates, formates, tartrates, maleates, malates, citrates, benzoates, salicylates, ascorbates and mesylates. Each salt comprising a weak base cation and individual anions and all groups and subgroups of anions are particular embodiments of the invention.

Also provided are pharmaceutical compositions comprising a salt of a weak base compound of formula: 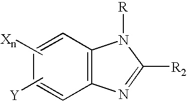
wherein X is hydrogen, halogen, alkyl of less than 7 carbon atoms or alkoxy of less than 7 carbon atoms; n is a positive integer of less than 4; Y is hydrogen, chlorine, nitro, methyl, ethyl or oxychloro; R is hydrogen, alkylaminocarbonyl wherein the alkyl group has from 3 to 6 carbon atoms or an alkyl group having from 1 to 8 carbons and R₂ is 4-thiazolyl, NHCOOR₁ wherein R₁ is aliphatic hydrocarbon of less than 7 carbon atoms, or an alkyl group of less than 7 carbon atoms; one or more free acids; and optional pharmaceutical additives. In particular embodiments, the salt and free acids are present in the composition at a ratio of about 1:0.5 to about 1:3 by weight. All individual values and ranges of ratios are included herein, including about 1:1 and about 1:2. Also provided are methods of making and using the salts and compositions described herein. Compositions consisting essentially of the components described herein are also included.

Also provided are methods of treating disease comprising administering to a patient a pharmaceutically effective amount of a pharmaceutical composition comprising a salt of a weak base compound of formula: 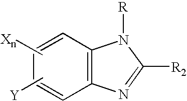
wherein X is hydrogen, halogen, alkyl of less than 7 carbon atoms or alkoxy of less than 7 carbon atoms; n is a positive integer of less than 4; Y is hydrogen, chlorine, nitro, methyl, ethyl or oxychloro; R is hydrogen, alkylaminocarbonyl wherein the alkyl group has from 3 to 6 carbon atoms or an alkyl group having from I to 8 carbons and R₂ is 4-thiazolyl, NHCOOR₁ wherein R₁ is aliphatic hydrocarbon of less than 7 carbon atoms, or an alkyl group of less than 7 carbon atoms; one or more free acids; and optional pharmaceutical additives. In particular pharmaceutical compositions, the salt and free acid are present in the composition at a ratio of about 1:0.5 to about 1:3 by weight. Other ratios as described herein are also included.

As used herein, “free acid” means a composition that ionizes in water to form hydrogen ion and an anion. In certain compositions of the invention, the free acid contains the same anion as the salt. In certain compositions of the invention, the free acid contains one or more anions, one of which can be the same anion as in the salt. As used herein, “salt” means a composition that ionizes in water to form an anion and a cation. In the salts of the invention, the weak base provides the cation in the salt.

As used herein, “weak base” or “weak bases” are those compounds having a pKa below about 7. Weak bases include prodrugs of weak bases. Preferred weak bases have a pKa below about 5. Other preferred weak bases have a pKa below about 4. Weak bases having pKa values below about 7 and compounds in all pKa ranges below about 7 are included in the invention. Some classes of weak bases include: imidazole derivatives having a pKa below about 7, pyridine derivatives having a pKa below about 7, aniline derivatives having a pKa below about 7 and compounds containing combinations thereof having a pKa below about 7. Imidazole derivatives are defined as compounds which include the structure:

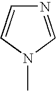

Some preferred imidazole derivatives include the following:

| Compound | pKa |
|---|---|
| Cimetadine | 6.8 |
| 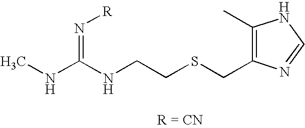 | |
| Glyodin | ˜7 |
| 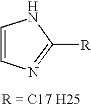 | |
| Miconazole | 6.7 |
| 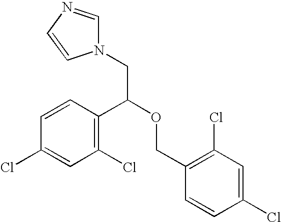 | |

Pyridine derivatives are defined as compounds which include the structure:

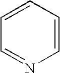

Some preferred pyridine derivatives include the following:

| Compound | pKa |
|---|---|
| Nicotinamide | 3.4 |
| 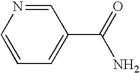 | |
| Nikethamide | 3.5 |
| 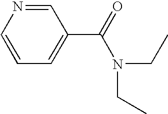 | |

Aniline derivatives are defined as compounds which include the structure: 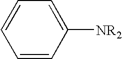
where R is hydrogen or alkyl having from 1 to 7 carbon atoms. The aromatic ring may have other substituents, as known in the art.

Some preferred aniline derivatives include the following:

| Compound | pKa |
|---|---|
| BPU NSC 639829 | ˜5 |
| 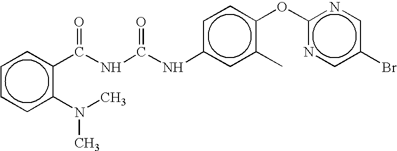 | |
| AMPB | 4 |
| 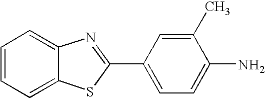 | |
| Minioxadil | 4.6 |
| 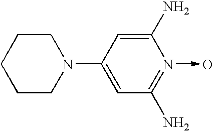 | |
| Benzocaione | 2.5 |
| 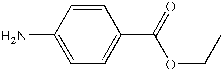 | |
| Butamben | 5.4 |
| 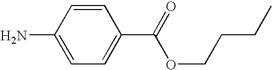 | |

One class of imidazole derivatives include those with the formula: 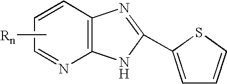
where n is an integer from 1 to 3, R is hydrogen, alkyl having from 1 to 7 carbon atoms, chloro, bromo, fluoro, oxychloro, hydroxy, sulfhydryl, or alkoxy having the formula —O(CH₂)_{y}(CH₃), wherein y is an integer from 0 to 6. One particular compound of this class is PG 300995:

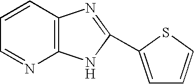

Another class of imidazole derivatives includes benzimidazoles and benzimidazole derivatives. As used herein, “benzimidazoles” are those having the formula: 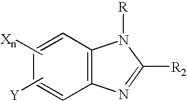
wherein X is hydrogen, halogen, alkyl of less than 7 carbon atoms or alkoxy of less than 7 carbon atoms; n is a positive integer of less than 4; Y is hydrogen, chlorine, nitro, methyl, ethyl or oxychloro; R is hydrogen, alkylaminocarbonyl wherein the alkyl group has from 3 to 6 carbon atoms or an alkyl group having from 1 to 8 carbons and R₂ is 4-thiazolyl, NHCOOR₁ wherein R₁ is aliphatic hydrocarbon of less than 7 carbon atoms, or an alkyl group of less than 7 carbon atoms. A preferred class of benzimidazoles are those wherein R is hydrogen. Another preferred class of benzimidazoles are: 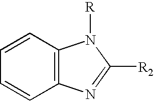
wherein R is an alkyl of 1 through 8 carbon atoms and R₂ is selected from the group consisting of 4-thiazolyl or NHCOOR₁ wherein R₁ is methyl, ethyl or isopropyl and pharmaceutically acceptable acid salts thereof with both organic and inorganic acids.

As used herein, “benzimidazole derivatives” include benzimidazoles as defined above, and prodrugs of benzimidazoles. “Prodrugs” are considered to be any covalently bonded carriers which release the active parent drug (weak base) according to the formula of the parent drug described above in vivo when such prodrug is administered to a mammalian subject. Prodrugs of the weak bases are prepared by modifying functional groups present in the compounds in such a way that the modifications are cleaved, either in routine manipulation or in vivo, to the parent compounds. Prodrugs include compounds wherein hydroxy, amine, or sulfhydryl groups are bonded to any group that, when administered to a mammalian subject, cleaves to form a free hydroxyl, amino, or sulfhydryl group, respectively. Examples of prodrugs include, but are not limited to, acetate, formate, or benzoate derivatives of alcohol and amine functional groups in the weak bases; phosphate esters, dimethylglycine esters, aminoalkylbenzyl esters, aminoalkyl esters and carboxyalkyl esters of alcohol and phenol functional groups in the weak bases; and the like.

The compositions of the invention are useful for administration to animals, preferably mammals, and preferably humans. The compositions of the invention are administered using any form of administration and any suitable dosage that provides a pharmaceutically active dose in an animal, preferably a mammal, as known in the art.

The compositions of the invention are used for oral, slow intravenous injection or infusion administration, as known in the art. Because the compositions are acidic, other forms of administration may be unsuitable. If the compositions are injected, the injection speed should be slow to avoid local irritation, as known in the art.

The compositions of the invention may be formulated as known in the art, and described in WO 01/12169, U.S. Pat. No. 3,903,297, and U.S. Pat. No. 6,423,734 for example, all of which are incorporated by reference to the extent not inconsistent with the disclosure herewith, and especially for details of formulations.

The compositions of the present invention may be administered in a unit dosage form and may be prepared by any method well known in the art without undue experimentation. Such methods include combining the compositions of the present invention with a carrier or diluent which constitutes one or more pharmaceutically acceptable additives, as known in the art without undue experimentation. Dosages of the compositions of the invention and frequency of adminstration are easily determined by means known in the art without undue experimentation.

Oral formulations suitable for use in the practice of the present invention include capsules, gels, cachets, tablets, effervescent or non-effervescent powders or tablets, powders or granules; as a solution or suspension in aqueous or non-aqueous liquid; or as an oil-in-water liquid emulsion or a water-in-oil emulsion. The compositions of the present invention may also be presented as a bolus, electuary or paste. Capsules or tablets can include suitable additives that provide desired properties, such as binders, lubricants, diluents, disintegrating agents, coloring agents, flavoring agents, flow-inducing agents and melting agents, as known in the art.

Techniques and compositions for making dosage forms useful in the present invention are described in the following references: 7 Modern Pharmaceutics, Chapters 9 and 10 (Banker & Rhodes, Editors, 1979); Lieberman et al., Pharmaceutical Dosage Forms: Tablets (1981); and Ansel, Introduction to Pharmaceutical Dosage Forms 2nd Edition (1976). Suitable pharmaceutical carriers are described in Remington's Pharmaceutical Sciences, Mack Publishing Company, a standard reference text in this field.

Also provided are kits useful in treating disease, which comprise one or more compositions of the invention and may include instructions for administration.

“Pharmaceutically acceptable” and “non-toxic” mean suitable for use with humans and/or animals without undue adverse side effects (such as toxicity, irritation and allergic response) commensurate with a reasonable benefittrisk ratio. “Pharmaceutically active” means capable of causing an intended physiological change in an animal, preferably a mammal.

“Pharmaceutically acceptable additives” include cosolvents, surfactants, complexants, hydrotropes and other components that are desired for pharmaceutical use, as known in the art, such as pharmaceutically acceptable carriers, preservatives, emulsifying agents, diluents, sweeteners, flavorants, viscosity controlling agents, thickeners, colorants and melting agents. Any level of pharmaceutically acceptable additives and any individual pharmaceutically acceptable additive or combination of additives may be used, as long as these additives do not reduce the solubility below a desired level or make the composition toxic, as defined above. The term “pharmaceutically acceptable carrier” is known in the art, see, for example, U.S. Pat. No. 6,479,526.

As used herein, “about” is intended to indicate a range caused by experimental uncertainty. When used in conjunction with ratios of salts and acids, “about” means ±5%.

As used herein, “patient” means an animal, mammal or human. One class of patients is mammals. One class of patients is human.

### BRIEF DESCRIPTION OF THE DRAWINGS

FIG. 1 shows microscopic pictures of different salts of carbendazim.

FIG. 2 shows x-ray powder patterns for different salts of carbendazim.

FIG. 3 shows DSC thermograms of different salts of carbendazim.

FIG. 4 shows HSM photographs of carbendazim sulfate.

FIG. 5 shows TGA thermograms of carbendazim sulfate and carbendazim hydrochloride.

FIG. 6 shows packing arrangement of sulfate salt along b-axis.

FIG. 7 shows TGA thermograms of carbendazim hydrochloride at different heating rates.

FIG. 8 shows logarithm of heating rates versus reciprocal absolute temperature; Δ: C=0.0799 and ⋄: C=0.05.

FIG. 9 shows thermal ellipsoid plot of molecules of different salts of carbendazim in the asymmetric unit at 50% probability, showing atomic numbering scheme: (a) hydrochloride; (b) phosphate; (c) sulfate; (d) mesylate; (e) besylate; and (f) tosylate.

FIG. 10 shows helix type arrangement around a two fold screw axis; a: carbendazim moieties; b: hydrochloride salt.

FIG. 11 shows packing arrangement of the phosphate salt along c-axis.

FIG. 12 shows packing arrangement of the sulfate salt along b-axis.

FIG. 13 shows packing arrangement of mesylate salt along b-axis.

FIG. 14 shows packing arrangement of besylate salt along b-axis.

FIG. 15 shows packing arrangement of tosylate against a two fold axis along c-axis.

FIG. 16 shows moisture adsorption curves for different salts of carbendazim: □: hydrochloride salt; x: sulfate salt; ⋄: tosylate salt; *: besylate salt; Δ: phosphate salt; and ▪: mesylate salt.

FIG. 17 shows powder x-ray diffraction patters for: (a) mesylate salt and (b) phosphate salt.

FIG. 18 shows dissolution profiles of carbendazim and its salts in (a) water and (b) 0.1 N HCl; ∘: free base; ⋄: hydrochloride salt; x: phosphate salt, ▪: sulfate salt, ♦: mesylate salt; □: besylate salt; and ▴: tosylate salt.

FIG. 19 shows dissolution profiles of phosphates in water; o: free base, *: physical mixture (1:1), x: phosphate salt, and Δ: physical mixture (1:2).

FIG. 20 shows dissolution profiles of tosylates in water; ▴: tosylate salt, Δ: equimolar physical mixture of tosylate salt—p-toluenesulfonic acid.

### DETAILED DESCRIPTION OF THE INVENTION

The invention may be further understood by reference to the following non-limiting examples. One of ordinary skill in the art will appreciate that all weak bases and acids other than those particularly exemplified can be used without undue experimentation. Applicant does not wish to be bound by any theory presented herein.

Synthesis

Weak bases, including benzimidazole derivatives are commercially available or can be prepared in a number of ways well known to one skilled in the art of organic synthesis without undue experimentation. The benzimidazole derivatives are synthesized using the methods described below, together with synthetic methods known in the art of synthetic organic chemistry, or variations thereon as appreciated by those skilled in the art without undue experimentation.

Benzimidazole derivatives may be prepared according to the method described in U.S. Pat. No. 3,738,995 issued to Adams et al, Jun. 12, 1973. The thiazolyl derivatives may be prepared according to the method described in Brown et al., J. Am. Chem. Soc., 83 1764 (1961) and Grenda et al., J. Org. Chem., 30, 259 (1965).

Materials

Carbendazim was provided by the Procter & Gamble Company (Cincinnati, Ohio) and used as received. All other chemicals were of reagent grade, purchased from Sigma (St. Louis, Mo.) or Aldrich (St. Louis, Mo.) and used without further purification.

Salt Preparation

The major issue for salt selection of an ionizable drug is the consideration of the relative basicity (or acidity) of the drug and the relative strength of the conjugate acid (base). In order to form a salt, the pKₐ of the conjugate acid has to be at least two units less than the pKₐ of the basic centre of the drug. Preferably, the selected counter-ion should possess minimal toxic effects. Carbendazim, has basic pKₐ of 4.5. The following anionic counter-ions were used for salt preparation:

TABLE 1

| Acid | Mol Wt | Pkₐ₁ | Pkₐ₂ | pKₐ₃ |
|---|---|---|---|---|
| HCl | 36.46 | <−6 | | |
| H₂S0₄ | 98.08 | −3 | | |
| p-Toluenesulfonic acid | 172.21 | −1.34 | | |
| Methanesulfonic acid | 96.10 | −1.2 | | |
| Benzenesulfonic acid | 158.18 | 0.7 | | |
| Phosphoric acid | 98.0 | 1.96 | 7.12 | 12.32 |

Phosphoric acid (0.98 g) was added to 100 ml of water kept on a heating plate maintained at 70° C. To this solution, 1.92 g of carbendazim was then added portionwise. On reacting to form the salt, carbendazim started dissolving. The system was heated to favor the reaction and to increase the solubility of the formed salt. The slurry was constantly stirred at 250 rpm for about 60 min until a saturated solution was obtained. The saturated solution was vacuum filtered immediately using a preheated (70° C.) glass filter into a conical flask that was preequilibrated at (70° C.). The final filterate was cooled slowly to room temperature by putting it back on the heating plate programmed to 2° C./min decrease in temperature. The solution was then left at room temperature for overnight, whereby needle shaped crystals crashed out of the solution. The formed crystals were removed from the water using a spatula and dried on filter paper to ensure evaporation of surface water molecules.

Similarly, other salts (hydrochloride, sulfate, tosylate, and besylate) were prepared wherein equimolar amount of acid and free base were added. To synthesize the mesylate salt the procedure must be modified due to high solubility of the free base in methanesulfonic acid. To a 2 ml solution of 2M methanesulfonic acid, 600 mg of carbendazim was added portionwise and vortexed. The suspension was then rotated overnight on an end-to-end rotator. Then, it was filtered and left at room temperature to evaporate slowly for 2 days, when fine needle like crystals were obtained. The crystals were separated from the solution by filteration and washed with isopropyl alcohol to rid of excessive methanesulfonic acid. The crystals were then air dried to ensure evaporation of isopropyl alcohol.

Thermal Analysis

Thermal analysis methods used included differential scanning calorimetry (DSC), thermogravimetric analysis (TGA), and hot stage microscopy (HSM). DSC traces were recorded with a TA Instruments DSC Q1000 (TA Instruments, New Castle, Del.). Samples weighing 1-3 mg were heated in crimped aluminum pans at a rate of 5° C./min under nitrogen flow of 40 ml/min. TGA analysis was performed on all samples that were indicated by the DSC as being possible solvates or hydrates.

TGA traces were recorded with a TA Instruments TGA Q-50 (TA Instruments, New Castle, Del.). The sample weight was approximately 2-4 mg, and heating rates of 2-15° C./min under nitrogen gas flow of 60 ml/min were used.

HSM analysis was carried out on small amounts of sample with a Mettler FP 82 hot stage equipped with a Mettler FP 80 central processor (Mettler, Columbus, Ohio), focused on Leica DM LP microscope (E. Licht Co., Denver, Colo.). The effects of temperature increase on the crystal behavior of samples were studied by placing a small amount of each sample on a glass slide, covering it with a coverslip, and gradually increasing the temperature to about 300° C. at a heating rate of 10° C./min.

Dehydration was observed with samples immersed in mineral oil. Photographs were taken using a Nikon 100 Nic digital camera equipped with a Diagnostics Instruments 1X-HRD digital camera coupler (Diagnostics Instruments Inc., Sterling Heights, Mich.) and transferred to a computer.

Powder X-Ray Diffraction (PXRD)

The PXRD patterns of different salts of carbendazim were determined at ambient temperature and atmosphere using a Philips PM 990/100 diffractometer (Philips, The Netherlands). The x-ray generator (PW3373/00 Cu LFF DK119706) has a copper radiation source which generates a voltage of 50 kV, and a current of 40 mA. Counts were measured using a X'Celerator detector which is based on real time multiple strip (RTMS) technology. Samples were packed into zero background silicon sample holders, and precautions were used to avoid introducing preferred orientation of the crystallites. The samples were subjected to a spinning movement having a rotation time of 4 s. The samples were scanned with the diffraction angle, 2θ, increasing from 3° to 63°, with a step size of 0.0167° and a counting time of 15.24 s. The XRD pattern traces of samples (salts subjected to moisture sorption or stability) were compared with regard to peak position and relative intensity, peak shifting, and the presence or lack of peaks in certain angular regions.

Moisture Sorption Studies

The moisture sorption of the various salts was determined by exposing weighed amounts (˜2 to 3 mg) of salts in a 4 ml glass vial, which was placed in sealed desiccators containing saturated salt solutions. Saturated salt solutions that give defined relative humidities (as a function of temperature) have been reported in various handbooks containing physical and chemical data.

The current study was performed at 25° C. with relative humidity values of 43% (saturated solution of potassium carbonate) and 81% (saturated solution of potassium bromide). The samples were stored in desiccators of known relative humidity for 8 days, following which they were re-weighed to calculate the % weight change. The solid phases were then analyzed using PXRD to ascertain the effect of the moisture content.

Solubility Determination

A quantity of each crystalline carbendazim salt which exceeded the amount required to yield a saturated solution with respect to the salt (unless solubility exceeded 1 M) was rotated 4 to 7 days in a 4 ml glass vial containing 1-2 ml of Millipore water at room temperature. The solubility of salts in water at 37° C. and 45° C. were determined by placing the vials in calibrated constant temperature water baths (Jouan Inc., Winchester, Va.) held within 0.05° C. of the temperature of the run. These were mixed by end-to-end rotation. Each sample was then filtered through a 0.45 μm PVDF filter. The filterate was collected in two or more fractions, which were analyzed separately by HPLC to ensure that there were no misleading solubility measurements resulting from adsorption onto the filter. Filter adsorption was assumed to be negligible when the concentration of successive fractions agreed within ±5%. The composition of the residual solid was examined to ensure that at least some of the solid phase that was in equilibrium with the solution was indeed the salt. The solubility of the different salts was also determined in 0.01M and 0.1M of the corresponding acids.

The Kₛₚ determinations were generally calculated directly from the observed carbendazim concentration once it was established that the residual solid phase contained excess salt. Since free acid precipitation results in the neutralization of an equivalent amount of counterion to its free base form, the same procedure could be used to calculate Kₛₚ in systems where free acid precipitation occurred prior to saturation of the system with the given salt.

Dissolution Studies

The dissolution of carbendazim and its salts at room temperature were studied in Millipore water and 0.1N hydrochloric acid (HCl) at pH 1.09. The dissolution of physical mixtures of drug and phosphoric acid in the molar ratios of 1:1 and 1:2 was also studied. A weighed amount of salt (so as to have 50 mg of carbendazim) was triturated to provide a uniform particle size and was suspended in the dissolution media. The volume of dissolution media was 250 ml, and the stirring rate was maintained at 250 rpm. One ml aliquot samples filtered through a 0.45 μm Millipore filter were withdrawn at 1, 5, 10, 15, 20, 30, 45, and 60 min, respectively. One ml of the dissolution medium was added to the dissolution vessel after each sampling period to maintain constant volume. The samples were then analyzed by HPLC.

High Performance Liquid Chromatography

A Beckman Gold HPLC system equipped with a model no. 168 detector at 280 nm was used for all assays. A Pinnacle ODS amine column (250×4.6 mm, Restek, Bellefonte, Pa.) was used with a mobile phase composed of 40% 20 mM phosphate buffer at pH 3 and 60% acetonitrile. The flow rate was controlled at 0.8 ml/min, with carbendazim's retention time at 3.5 min. The injection volume was 20 μL. The evaluation of the assay was conducted by using carbendazim standard solutions at concentrations ranging from 0.1 μg/ml to 100 μg/ml. None of the solubilization agents interfered with the assay. All experimental data are the average of duplicate values with an average error less than 3%.

Single Crystal X-Ray Structural Analysis

A colorless block of carbendazim phosphate having approximate dimensions of 0.07×0.22×0.37 mm was mounted on a glass fiber in a random orientation. Examination of the crystal on a Bruker SMART 1000 CCD detector X-ray diffractometer at 443(2)K and a power setting of 50 KV, 40 mA showed measurable diffraction to at least θ=24.4565°. Data were collected on the SMART1000 system using graphite monochromated Mo K radiation (=0.71073 Å).

Initial cell constants and an orientation matrix for integration were determined from reflections obtained in three orthogonal 5° wedges of reciprocal space. A total of X frames at 1 detector setting covering 0<2θ<60 deg were collected, having an o scan width of 0.3 and an exposure time of 10 seconds. The frames were integrated using the Bruker SAINT software package's narrow frame algorithm. Out of the 7239 total reflections that were integrated and retained, 2676 were unique (redundancy=2.7, Rint=2.8%, Rsig=3.2%). Of the unique reflections, 2287 (85.5%) were observed with I>2σ(I). The final Triclinic cell parameters of a=7.7610(9)Å, b=9.0368(11)Å, c=9.9799(11)Å, a=115.098(2), β=104.913(2), γ=98.536(2), volume=585.36(12) Å³ are based on the refinement of the XYZ-centroids of 3034 reflections with I>3σ(I), covering the range of 2.4125<θ<24.4565. Empirical absorption and decay corrections were applied using the program SADABS. The absorption coefficient is 0.265 mm⁻¹, Tₘᵢₙ=0.9084, and Tₘₐₓ=0.9817. For Z=2 and formula weight (FW)=289.19, the calculated density is 1.641 g/cm³. Systematic absences and intensity statistics indicate the space group to be P 1̅ (#2) which was consistent with refinement.

The structure was solved using SHELXS in the Bruker SHELXTL (Version 5.0) software package. Refinements were performed using SHELXL and illustrations were made using XP. Solution was achieved utilizing direct methods followed by Fourier synthesis. Hydrogen atoms were added at idealized positions, constrained to ride on the atom to which they are bonded, and having thermal parameters equal to 1.2 or 1.5 times Uᵢₛₒ of that bonded atom. The final anisotropic full-matrix least squares refinement based on the F² of all reflections converged (maximum shift/esd=0.000) at R₁=0.0421, wR₂=0.0987 and goodness-of-fit=1.060. “Conventional” refinement indices using the 2287 reflections with F>4σ(F) are R₁=0.0349, wR₂=0.0941. The model consisted of 220 variable parameters, 0 constraints and 0 restraints. There were 24 correlation coefficients between 0.555 and 0.633 due to the obtuse angle, which involve off-diagonal thermal parameters. The highest peak on the final difference map was 0.468 eA⁻³, located 0.66 from C(8). The lowest peak occurred at −0.303 eA⁻³, located 0.59 from P(1). Scattering factors and anomalous dispersion were taken from International Tables Vol C, Tables 4.2.6.8 and 6.1.1.4.

The single crystal x-ray structure determination for all the other salts were carried out in a manner similar to that discussed above. The data are summarized in Table 2.

RESULTS AND DISCUSSION

Morphology of the Carbendazim Salts

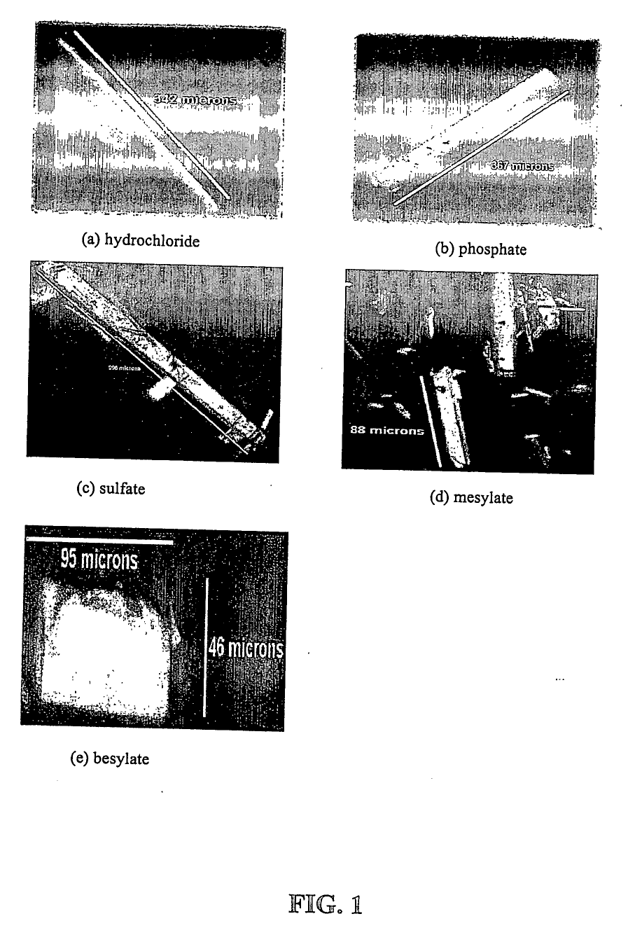

After preparation and recovery of the crystal forms, visual and microscopic evaluation (FIG. 1) clearly showed differences in the morphology of the prepared salts from the original compound. Using microscopy, all of the salts examined where characterized as either monoclinic or orthorhombic.

X-Ray Diffraction Analysis

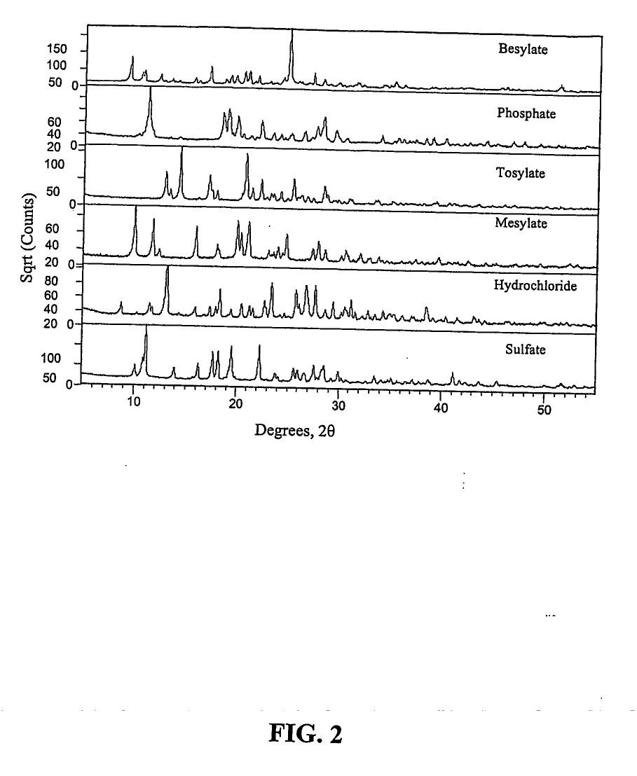

Each of the prepared salts had a distinct characteristic PXRD pattern, as shown in FIG. 2. These PXRD patterns were compared with the respective samples that were subjected to moisture sorption studies or stability studies.

TABLE 2
Crystallographic data for single x-ray crystal structure of different carbendazim salts

| | Hydrochloride | Phosphate | Sulfate | Tosylate | Benzoylate | Mesylate |
|---|---|---|---|---|---|---|
| Crystal data | | | | | | |
| Empirical | C₉H₁₄ClN₃O₄ | C₉H₁₂N₃O₆P | C₁₈H₂₄N₆O₁₀S | C₁₆H₁₇N₃O₅S | C₁₅H₁₅N₃O₅S | C₁₀H₁₃N₃O₅S |
| formula | | | | | | |
| Formula | 263.68 | 289.19 | 516.49 | 363.39 | 349.36 | 287.29 |
| weight | | | | | | |
| Crystal size | 0.55 × 0.12 × | 0.37 × 0.22 × | 0.25 × 0.12 × 0.04 | 0.34 × 0.09 × 0.08 | 0.39 × 0.35 × 0.18 | |
| (mm³) | 0.08 | 0.07 | | | | |
| Crystal system | Orthorhombic | Triclinic | Monoclinic | Orthorhombic | Triclinic | Monoclinic |
| Space group | P2₁2₁2₁ | P1 | C2/c | P2₁2₁2₁ | P1 | Cc |
| Unit cell | a = 5.6916(7) | a = 7.7610(9) | a = 19.666(4) | a = 7.6805(7) | a = 9.0068(5) | a = 5.1602(8) |
| dimensions (Å) | b = 13.3375(17) | b = 9.0368(11) | b = 6.7980(15) | b = 13.4035(12) | b = 9.3944(5) | b = 17.688(3) |
| | c = 15.4889(19) | c = 9.9799(11) | c = 18.250(4) | c = 15.9194(14) | c = 9.6643(6) | c = 14.321(2) |
| | α = 90° | α = 115.098° | α = 90° | α = 90° | α = 87.9450° | α = 90° |
| | β = 90° | β = 104.913° | β = 115.687° | β = 90° | β = 75.7680° | β = 98.150° |
| | γ = 90° | γ = 98.536° | γ = 90° | γ = 90° | γ = 74.9240° | γ = 90° |
| Volume (Å³) | 1175.8(3) | 585.36(12) | 2198.7(8) | 1638.8(3) | 765.01(8) | 1293.9(3) |
| Z | 4 | 2 | 8 | 4 | 2 | 4 |
| Calculated | 1.490 | 1.641 | 1.560 | 1.473 | 1.517 | 1.475 |
| density | | | | | | |
| (mg/m³) | | | | | | |
| Absorption | 0.333 | 0.265 | 0.218 | 0.231 | 0.244 | 0.271 |
| coefficient | | | | | | |
| (mm⁻¹) | | | | | | |
| F(000) | 552 | 300 | 1080 | 760 | 364 | 600 |
| Data Collection | | | | | | |
| Diffractometer | Bruker SMART | Bruker SMART | Bruker SMART | Bruker SMART | Bruker SMART | Bruker SMART |
| | 1000 CCD | 1000 CCD | 1000 CCD | 1000 CCD | 1000 CCD | 1000 CCD |
| Temperature | 170 | 170 | 170 | 170 | 170 | 170 |
| (K) | | | | | | |
| Wavelength | 0.1707 | 0.7107 | 0.7107 | 0.7107 | 0.7107 | 0.7107 |
| (Å) | | | | | | |
| θ range for | 2.01-26.11° | 2.41-27.54° | 2.30-27.52° | 1.99-25.55° | 2.18-27.90° | 2.30-26.03° |
| data collection | | | | | | |
| Index ranges | −7 ≦ h ≦ 7, | −10 ≦ h ≦ 10, | −25 ≦ h ≦ 25, | −9 ≦ h ≦ 9, | −11 ≦ h ≦ 11, | −6 ≦ h ≦ 6, |
| | −16 ≦ k ≦ 16, | −11 ≦ k ≦ 11, | −8 ≦ k ≦ 8, | −16 ≦ k ≦ 16, | −12 ≦ k ≦ 12, | −21 ≦ k ≦ 21, |
| | −18 ≦ l ≦ 19 | −12 ≦ l ≦ 12 | −23 ≦ l ≦ 23 | −19 ≦ l ≦ 19 | −12 ≦ l ≦ 12 | −17 ≦ l ≦ 17 |
| Reflections | 12994 | 7239 | 13030 | 17552 | 9724 | 7047 |
| collected | | | | | | |
| Independent | 2321 | 2676 | 2540 | 3062 | 3619 | 2569 |
| reflections | | | | | | |
| Solution and | | | | | | |
| Refinement | | | | | | |
| System used | SHELXTL- | SHELXTL- | SHELXTL- | SHELXTL- | SHELXTL- | SHELXTL- |
| | V5.0 | V5.0 | V5.0 | V5.0 | V5.0 | V5.0 |
| Solution | Direct methods | Direct methods | Direct methods | Direct methods | Direct methods | Direct methods |
| Refinement | Full-matrix | Full-matrix | Full-matrix | Full-matrix | Full-matrix | Full-matrix |
| method | least-squares on | least-squares on | least-squares on | least-squares on | least-squares on | least-squares on |
| | F² | F² | F² | F² | F² | F² |
| Max/min | 0.9732 and | 0.9817 and | 0.9913 and | 0.9817 and | 0.9580 and | |
| transmission | 0.8380 | 0.9084 | 0.9476 | 0.9255 | 0.9107 | |
| Absolute | −0.03(7) | | | 0.01(9) | | 0.32(8) |
| structure | | | | | | |
| parameter | | | | | | |
| Data/ | 2321/0/170 | 2676/0/220 | 2540/0/195 | 3062/0/226 | 3619/0/217 | 2569/2/173 |
| restraints/ | | | | | | |
| parameters | | | | | | |
| R indices | R₁ = 0.0397, | R₁ = 0.0349, | R₁ = 0.0419, | R₁ = 0.0433, | R₁ = 0.0369, | R₁ = 0.0384, |
| (I > 2σ(I)) | wR₂ = 0.0714 | wR₂ = 0.0941 | wR₂ = 0.0954 | wR₂ = 0.0828 | wR₂ = 0.0899 | wR₂ = 0.0892 |
| R indices (all | R₁ = 0.0523, | R₁ = 0.0421, | R₁ = 0.0738, | R₁ = 0.0538, | R₁ = 0.0509, | R₁ = 0.0437, |
| data) | wR₂ = 0.0754 | wR₂ = 0.0987 | wR₂ = 0.1098 | wR₂ = 0.0860 | wR₂ = 0.0933 | wR₂ = 0.0915 |
| Goodness of fit | 1.073 | 1.060 | 1.041 | 1.084 | 0.977 | 1.062 |
| on F² | | | | | | |
| Largest | 0.301 and −0.188 | 0.468 and −0.303 | 0.290 and −0.380 | 0.318 and −0.256 | 0.366 and −0.364 | 0.286 and −0.224 |
| difference peak | | | | | | |
| and hole (eÅ⁻³) | | | | | | |

Thermal Analysis

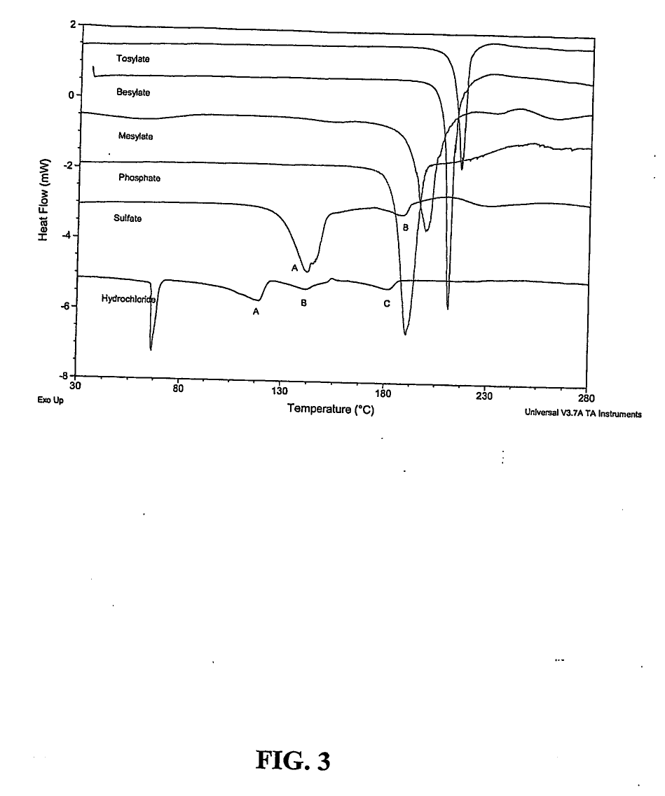

A summary of the DSC data is given in Table 3, which includes all thermal events (i.e., dehydration and melting) and their corresponding heat requirements. The DSC traces of each salt (FIG. 3), except hydrochloride and sulfate, showed a single melting endotherm, indicating that they were synthesized as anhydrous salts. On the other hand, the DSC traces of sulfate and hydrochloride showed more than one endotherm, suggesting the presence of the solvent molecule and/or polymorphs. Since water was the only solvent used in every salt preparation, it was believed that the above two salts were hydrates.

TABLE 3
DSC data of different salts of carbendazim with respect to thermal events
at a certain temperature with the corresponding heat required.

| | | | Melting Point | | |
|---|---|---|---|---|---|
| | Dehydration | | Endotherm (° C.) | | Heat of |
| Carbendazim | Endotherm | Heat | Onset | | Fusion |
| Salt | (° C.) | (J/g) | Temp | Peak Temp | (J/g) |
| Hydrochloride | 66.52 | 124.8 | 96.75 | 118.76 | 219.4 |
| Phosphate | — | — | 180.23 | 189.54 | 296.9 |
| Sulfate | — | — | 124.04 | 137.31 | 323.6 |
| Mesylate | — | — | 198.48 | 202.45 | 137.8 |
| Besylate | — | — | 208.17 | 211.23 | 171.0 |
| Tosylate | — | — | 213.18 | 216.47 | 162.7 |

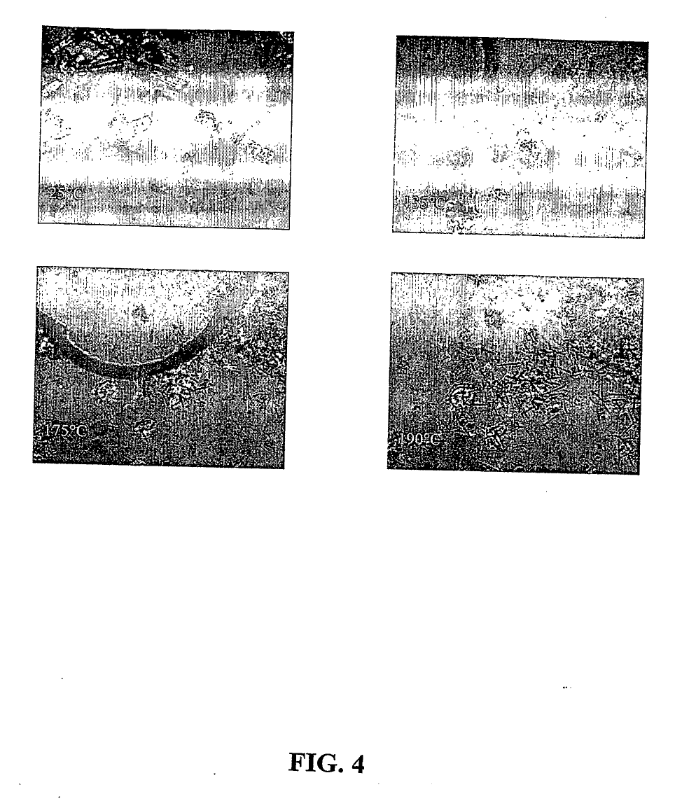

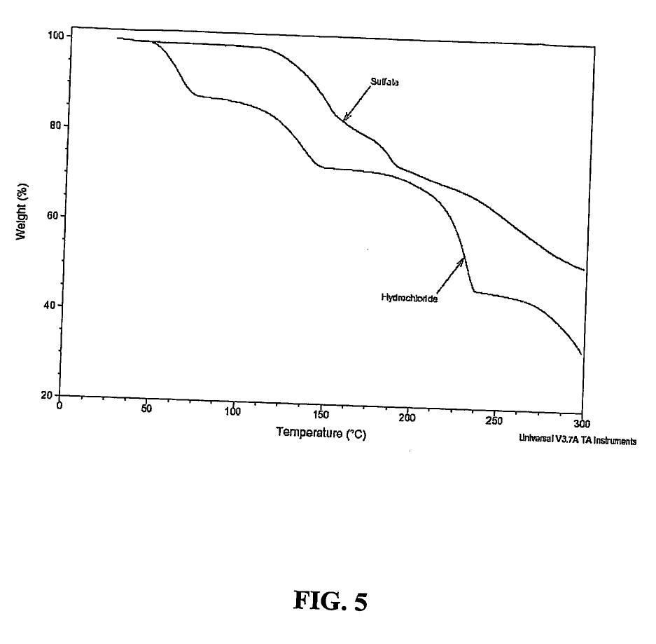

Hot stage microscopy (HSM) was used to ascertain different thermal events that were shown in the DSC traces of the hydrochloride and the sulfate salt. FIG. 4 shows the sequence of events recorded while heating a sample of carbendazim sulfate. Upon heating, the first endotherm (A), at 135° C. in the DSC trace of the sulfate salt probably corresponds to the melt of the salt accompanied by dehydration of the hydrated salt. The formation of air bubbles indicates liberation of water molecules, and shrinkage of molecules represents the melt. The basis for this endotherm was further investigated by using TGA. The TGA scan shown in FIG. 5 indicates a weight loss of about 19% over the temperature range of 120-170° C. This weight loss is more than the theoretical weight loss of 7%, calculated for a solvate consisting of two molecules of carbendazim, two molecules of water and a single molecule of sulfate. Therefore, the first endotherm should represent the melt of the hydrate.

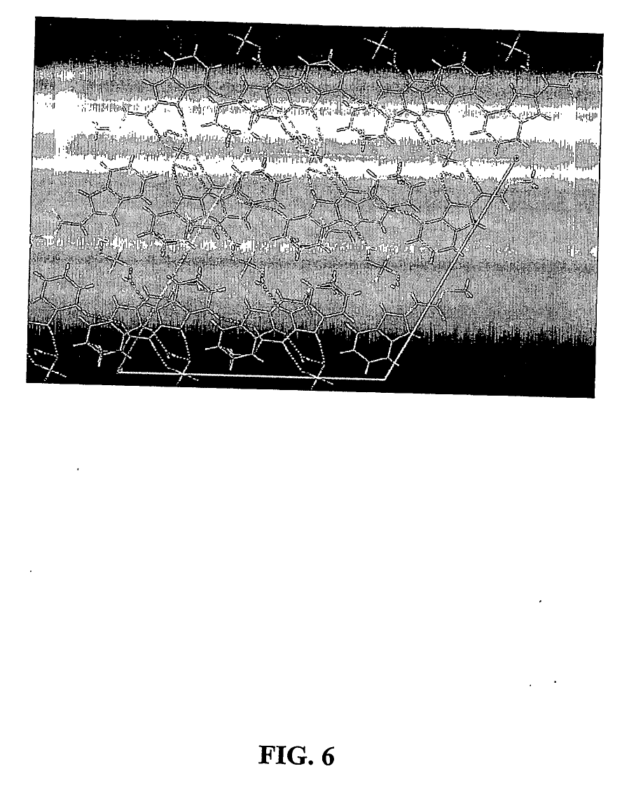

As the sample is heated further, a second form of the compound recrystallizes from the melt; however, this event was not detected by DSC analysis. The melt of this form corresponds to the second, smaller endotherm (B) on the DSC thermogram, and occurs around 175° C. In order to verify the assumption regarding the second form of the salt, the sulfate salt was heated in an oven maintained at 140° C., then the sample was run on the HPLC using the procedure previously described to check for the presence of carbendazim. The eluent at 3.5 min had a uv spectra similar to the original compound, thereby validating the assumption. After the second melt, recrystallization again occurs to form dendritic crystals. This dendritic crystal continues to grow until it melts, indicated by the third endotherm on the DSC trace, which happens to be the degradation product. The temperature of the first endotherm exceeds the boiling point of water by 35° C., which indicates formation of a stable ionic hydrate. This is confirmed by a detailed study of its packing arrangement, wherein the presence of water molecules helps in formation of a number of hydrogen bonds. The guest water molecules in the sulfate salt are located in isolated cavities along the length of the b-axis, forming H-bonds with sulfate, carbendazim and other water molecules. Dehydration of the crystal must therefore involve complete disruption of the crystal structure, as shown in FIG. 6, and should occur at a relatively high temperature owing to strong host-guest hydrogen bonding, and location of guest molecules in the isolated cavities.

The DSC thermogram of sulfate, along with TGA and HSM confirmed that there are two forms of the sulfate salt. Although the synthesized salt contains only one form, it is only after the melt of A that the other form grows from the melt. From the DSC thermogram of the sulfate salt, it is difficult to distinguish between monotropy and enantiotropy. The interpretation of the DSC curve is facilitated by the Burger's enthalpy of fusion rule: if the higher melting form has lower melting enthalpy, both forms are related enantiotropically. Table 4 lists the melting point and enthalpy of fusion for the different forms of the sulfate and the hydrochloride salts. The melting enthalpy of the higher melting form B is lower than the melting enthalpy of A. Therefore, the two forms are enantiotropically related, however only form A is stable below the transition temperature. In a similar fashion, the hydrochloride salt was found to undergo dehydration at 66° C., followed by a melting endotherm at 120° C. The weight loss of 13.1% (FIG. 5) over the temperature range 45-86° C. agrees with the theoretical value of 13.6%, which was calculated for a solvate containing two molecules of water for each molecule of the hydrochloride salt. The hydrochloride salt was found to have three forms, which are also related enantiotropically.

TABLE 4
Physical properties of different forms of a sulfate and a hydrochloride salt

| | Sulfate salt | | Hydrochloride salt | | |
|---|---|---|---|---|---|
| Property | Form A | Form B | Form A | Form B | Form C |
| MP (° C.) | 137.31 | 177.56 | 118.76 | 151.63 | 188.73 |
| ΔH_{fusion} (J/g) | 323.60 | 48.05 | 219.40 | 65.01 | 65.26 |

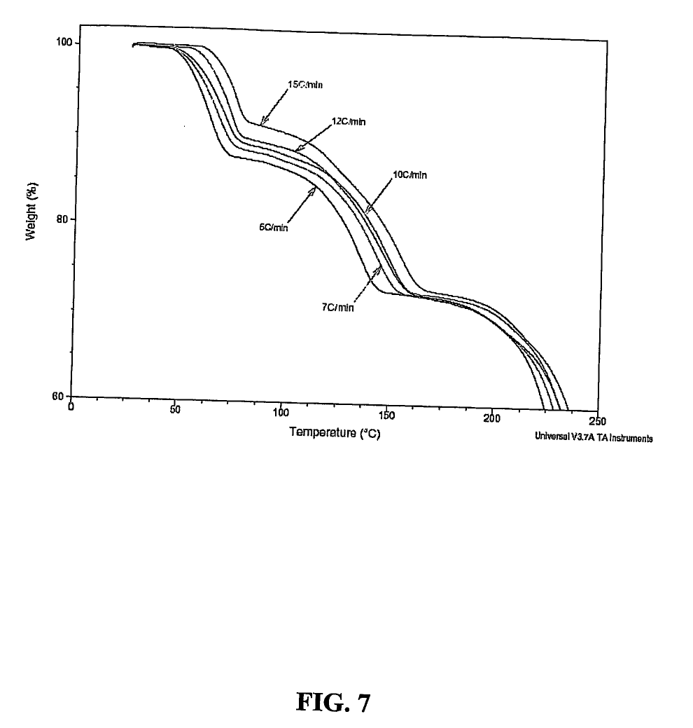

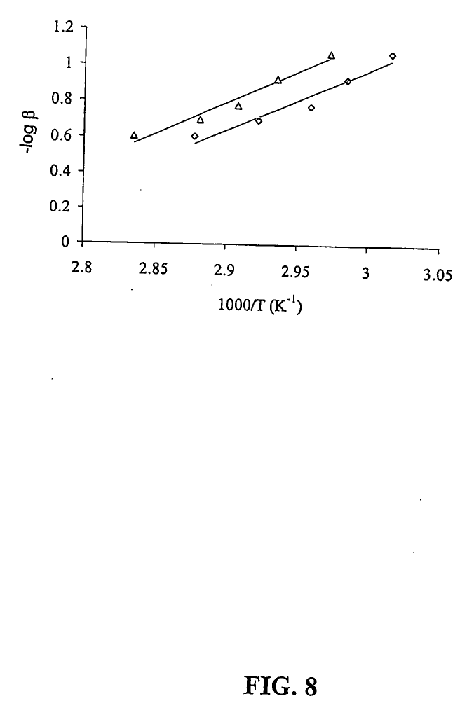

The dehydration kinetics of carbendazim hydrochloride dihydrate was studied by subjecting the crystals to TGA heating rates of 5, 7, 10, 12, and 15° C. per minute. TGA traces from that analysis are shown in FIG. 7. The activation energy (Eₐ) for the dehydration process was calculated from these TGA data according to the method described by Flynn and Wall (J. H. Flynn and L. A. Wall, J. Research Nat. Bur. Standards A, Phys. Chem. A71, 25 (1967); J. Polym. Sci., Pol. Left. 5, 191 (1967); J. Polym. Sci., Pol. Lett. 4, 323 (1966)). This method involves the analysis of weight loss versus temperature at different heating rates (β) to determine the corresponding absolute temperatures at a constant weight loss (C). Graphs of negative logarithm of heating rates (expressed in ° C./s) (−log β) versus 1/T were plotted (FIG. 8) and the activation energy calculated from the slope of the curves. The activation energy for the dehydration of the hydrochloride salt, calculated from the TGA data, was ˜64 kJ/mol.

Comparison of Structures of Different Carbendazim Salts

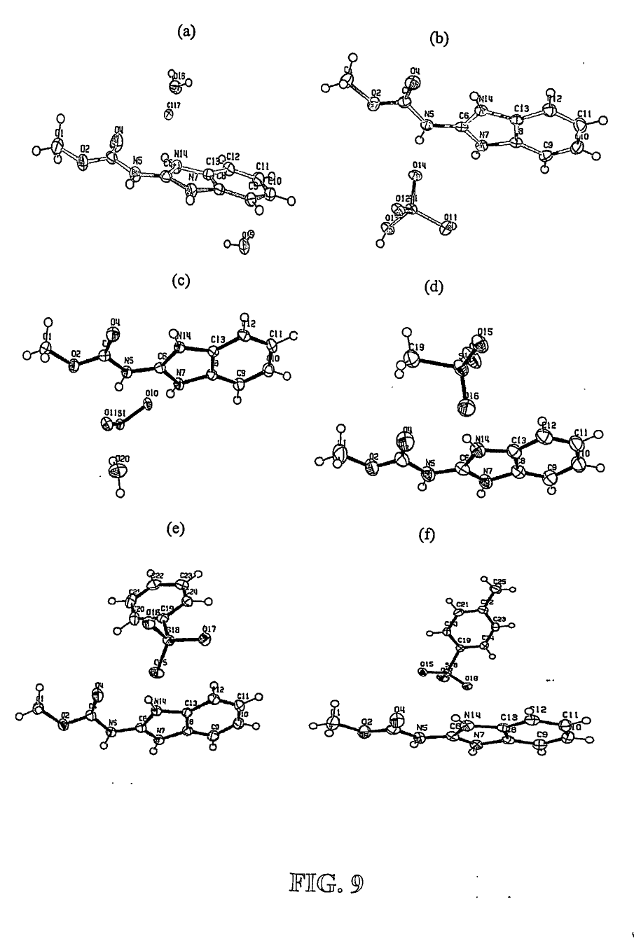

The X-ray crystal structure of different carbendazim salts permitted a detailed analysis of the conformational preferences, hydrogen-bonding interactions, and crystal packing forces that likely determine the physical properties of these crystal forms. Illustrations of these salts along with their atomic numbering are given in FIGS. 9(a-f). Final relevant atomic positions, bond lengths, bond angles, torsion angles, anisotropic thermal displacements and hydrogen positions are not given here.

In the carbendazim molecule, there is a double bond between C(3) and O(4) atoms (1.192(3) Å), whereas C(3) and O(2) are single bonded (1.335(3) Å). The bond distance between C(3) and O(2) is less than that for a single covalent bond value of 1.41 Å, suggesting that the O(2) atom has partial sp² characterization, thus making the C(1) of the methyl group less flexible.

The imidazole nitrogen N(14) is protonated, so the C(6)-N(14) bond is lengthened (1.332(3) Å). This proton is bonded, via intermolecular hydrogen bond, to the counter ion, for e.g. chloride ion CI(17) (N(14) . . . CI(17)=3.143 Å) in the hydrochloride salt. Although it is possible for the proton (H(14)) to have an intramolecular hydrogen bond with the oxygen O(4) of the carbamate group, though the bond angle is far from linear (∠N(14)H(14A)O(4)=116.12).

The positive charge of the carbendazim molecule is neutralized by the counter ion from the included acidic moiety. The formed cation is resonance stabilized, with the positive charge fluctuating among the three nitrogens N(5), N(7), and N(14). This is verified by the bond lengths of C(6)-N(5) (1.346(3)Å), C(6)-N(7) (1.338(3) Å), and C(6)-N(14) (1.332(3) Å), which are between the bond length values for a single C-N (0.143 Å) and a double C═N(0.127 Å) bond. The above information regarding the structure of carbendazim is true for all the formed salts.

The carbendazim moiety in all the studied salts was found to be arranged planar, irrespective of the crystal system/space group, and/or the counter-ion present in the crystal lattice. This is not surprising, as the presence of a benzimidazole ring on one end makes the molecule as a whole planar. Interestingly, the carbonyl group next to the oxygen of methoxy group imparts a slight sp² character to the oxygen, thereby restricting the free rotation of the methoxy group. This is confirmed by the inability of the oxygen to form a hydrogen bond with any of the available H—donors. None of the salts demonstrated intra or intermolecular hydrogen bonding between the carbendazim molecules, except for the sulfate salt, wherein there existed a weak C—H . . . O intermolecular hydrogen bond.

Apart from the normal covalent bonds, the packing arrangement of organic salts is mainly determined by its ability to form inter and intramolecular hydrogen bonds, and to a lesser extent, by van der Waals interactions. Thus, knowledge of hydrogen bond strength along with the hydrogen bond number (HBN) can be used in a qualitative fashion for correlations with melting point. It should be remembered that a compound's melting point is a function of several parameters, such as symmetry, eccentricity, packing, flexibity, and hydrogen bonding.

The strength of a hydrogen bond is a function of the electronegativity of the donor (D) and the acceptor (A) atoms. Since the closeness of the D and A atoms in a crystal is a measure of how effectively the hydrogen is acting as a mutual attractor, the crystallograghic A-B distances can be used as a measure of hydrogen bond strength. HBN is defined as the maximum number of hydrogen bonds that can exist in a repeating lattice. It is equal to twice the minimum of either the number of bondable hydrogens or the number of hydrogen bond acceptor sites on the molecule.

Although all the salts form multiple hydrogen bonds, only the prominent ones (based on strength) are listed in Table 5. All the sulfonic salts form reinforced hydrogen bonds in which 3 NHs on carbendazim are bonded with 3 Os on sulfonate and thus, have relatively higher melting points than all the others. However, as the sulfonate salts arrange themselves to form hydrogen bonds, they will, in theory, assume a packing arrangement which is less efficient than those of non-hydrogen bonding molecules. This is verified by the low values of packing efficiency for the sulfonate salts.

The phosphate salt also forms reinforced hydrogen bonds between the phosphate moieties, however, the carbendazim molecule is only moderately bonded to the phosphate, and therefore has melting point less than sulfonates, but greater than both the hydrochloride and the sulfate. On the other hand both the hydrochloride and the sulfate salt form a number of H-bonds (at least six), which may compete with each other and limit the formation of all bonds. It is very likely that the geometric constraints imposed by some H-bonds may severely inhibit additional bonds, resulting in low melting points. Because of less stringent requirements, both the sulfate and the hydrochloride salts are closely packed.

Interestingly, incorporation of solvent molecules into the crystal lattice appears to be positively or negatively related to attaining the maximum hydrogen bond number. The hydrochloride and sulfate salts are deficient in acceptor and donor atoms. The use of water as the solvent in these lattices is analogous to sharing electrons, allowing the hydrochloride and the sulfate to attain stable crystal structures.

TABLE 5
Comparison of major hydrogen bonds formed in crystal lattice of different
carbendazim salts based on criteria of hydrogen bond strength

| Donor (D) | | Acceptor (A) | | Δd^{a} (D . . . A) (Å) | | | | | |
|---|---|---|---|---|---|---|---|---|---|
| Molecule | Atom | Molecule | Atom | Hydrochloride | Phosphate | Sulfate | Mesylate | Besylate | Tosylate |
| CBZ | N (5) | Salt | O | 0.752 | 0.852 | 0.811 | 0.780 | 0.738 | 0.724 |
| CBZ | N (7) | Salt | O | 0.846 | 0.534 | 0.821 | 0.814 | 0.721 | 0.797 |
| CBZ | N (14) | Salt | O | | 0.815 | 0.710 | 0.765 | 0.713 | 0.698 |
| CBZ | N (14) | Salt | Cl | 0.827 | | | | | |
| Water | O | Salt | Cl | 0.880 | | | | | |
| Water | O | Salt | O | | | 0.590 | | | |
| Water | O | Salt | O | 0.469 | | 0.531 | | | |
| Phosphate | O (13) | Phosphate | O | | 0.907 | | | | |
| Phosphate | O (11) | Phosphate | O | | 0.877 | | | | |

| Properties | Hydrochloride^{b} | Phosphate | Sulfate^{c} | Mesylate | Besylate | Tosylate |
|---|---|---|---|---|---|---|
| Number of molecules in the unit cell | 4 | 2 | 8 | 4 | 2 | 4 |
| V_{vdw} (Å³)^{d} | 206 | 202 | 200 | 208 | 258 | 273 |
| V_{cell} (Å³)^{e} | 1176 | 585 | 2199 | 1294 | 765 | 1639 |
| Packing Efficiency (κ) | 0.70 | 0.69 | 0.73 | 0.64 | 0.68 | 0.67 |
| MP (° C.) | 119 | 190 | 137 | 202 | 211 | 216 |
| ΔH_{fusion} (J/g) | 219 | 297 | 324 | 138 | 171 | 163 |

^{a}Δd = [covalent(D-H) + vdw(H . . . A)] − observed(D-H . . . A)
^{b}hydrochloride salt was found to be dihydrate
^{c}sulfate salt was found to be carbendazim hemisulfate monohydrate
^{d}van der Waals volume of the molecule in the asymmetric unit
^{e}volume of the unit cell calculated from the single x-ray crystal data

Carbendazim Hydrochloride

The hydrochloride salt of carbendazim is crystallized in the orthorhombic space group P2₁2₁2₁. This space group is chiral, and does not have any symmetry operations associated with inversion or mirror. Thus, it is devoid of a centre of symmetry and aptly defined as non-centrosymmetric. The symmetry operation for this space group involves both rotation and translation along a given axis, referred to as the screw axis. In this space group, three two-fold screw axes are present along the a, b, and c directions. Thus, 2₁2₁2 mean that the asymmetric unit moves ½ of a repeat unit along the three axes for each ½ of a revolution about that axis. Because of these symmetry operations, 4 equipoint transformations are generated (x,y,z; ½+x, ½−y, −z; −x, ½+y, ½−z; ½−x, −y, ½+z), thereby having the general position multiplicity of 4 in the unit cell. The asymmetric unit of the carbendazim hydrochloride salt contains one molecule each of hydrochloride and carbendazim, along with two water molecules. The presence of two water molecules in the unit pattern illustrates the importance of water during the crystallization process.

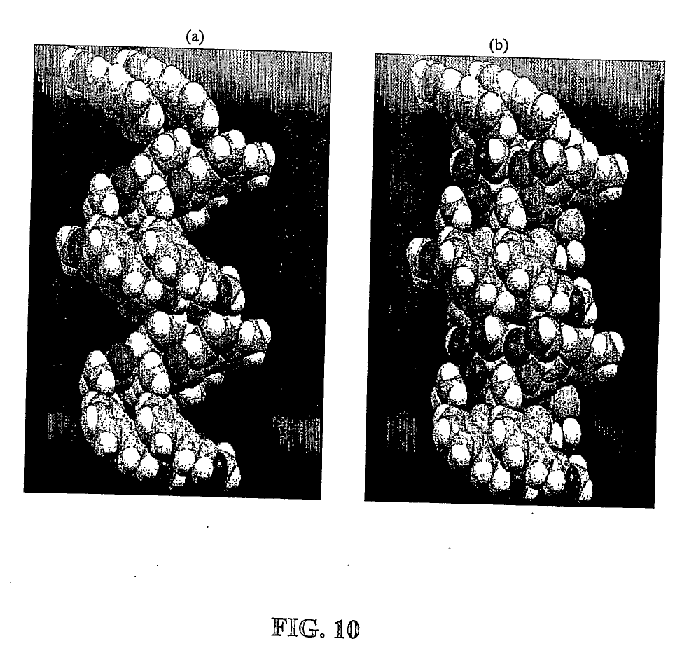

The thermal ellipsoidal diagram of the hydrochloride salt (FIG. 9(a)) includes the atomic labeling scheme, while the stereo packing diagram of its unit cell is shown in FIG. 10. The chloride anion along with the water molecules act as a cross linker for the carbendazim assemblies. The self-assembly patterns of carbendazim molecules (present as cationic species) arrange themselves in infinite helices around a two-fold screw axis, connecting through π-π stacking involving imidazole and a phenyl ring (FIG. 10(a)). Such assemblies create voids in which chloride ions and water molecules are contained (FIG. 10(b)). The chloride ions interact with the surrounding carbendazim ions and water molecules through multiple hydrogen bonds, one hydrogen bond with carbendazim (N(14)-H(14A) . . . CI(17)), and one hydrogen bond with each of the two water molecules (O(15)-H(15B) . . . CI(17) and O(16)-H(16A) . . . CI(17)). The water molecules also form hydrogen bonds with nitrogen (N(5) and N(7)) and oxygen (O(4)) of carbendazim ion. The two water molecules are also connected together through intermolecular hydrogen bonding.

A novel feature in the carbendazim hydrochloride assembly is the presence of C—H . . . CI bond formed between the C1(H1A) of the methyl group on the carbendazim and the chloride ion. The C(1)-H(1A) . . . CI(17) distance of 3.693 Å is not only less than the van der Waals distance of 4.08 Å but also it maintains remarkable linearity, the C—H . . . CI angle being 157.82°. The hydrogen bonding parameters for the hydrochloride salt are listed in Table 6. In the above discussed packing arrangement of hydrochloride salt, the Os on water act as both hydrogen bond acceptor and donor, whereas both N and C act as hydrogen donors and Cl as an acceptor.

TABLE 6
Geometrical parameters for hydrogen bonds observed in the
crystal lattice of carbendazim hydrochloride salt

| | d(H . . . A) | d(D . . . A) | ∠(D-H . . . A) |
|---|---|---|---|
| D-H . . . A | (Å) | (Å) | (°) |
| N(7)—H(7A) . . . O(15) | 1.786 | 2.664 | 174.79 |
| N(5)—H(5A) . . . O(16) | 1.886 | 2.758 | 170.74 |
| N(14)—H(14A) . . . Cl(17) | 2.365 | 3.143 | 147.66 |
| O(15)—H(15B) . . . Cl(17) | 2.297 | 3.070 | 160.82 |
| O(16)—H(16A) . . . Cl(17) | 2.395 | 3.205 | 173.74 |
| O(15)—H(15A) . . . Cl(17) | 2.456 | 3.249 | 151.04 |
| O(16)—H(16B) . . . Cl(16) | 2.398 | 3.082 | 149.07 |
| O(16)—H(16B) . . . Cl(15) | 2.491 | 3.021 | 127.06 |
| C(1)—H(1A) . . . Cl(17) | 2.767 | 3.693 | 157.82 |

D and A refer to donor and acceptor atoms, respectively.

The crystal structure is stabilized by numerous hydrogen bonds of the type N—H . . . O, N—H . . . Cl, O—H . . . O, O—H . . . Cl, and C—H . . . Cl. Apart from these bonds, all other intermolecular contacts correspond to normal van der Waals interactions.

Carbendazim Phosphate

The phosphate salt of carbendazim crystallizes in the triclinic system, having centrosymmetric space group, P 1̅. The triclinic crystal systems have no restrictions regarding cell edges and cell angles. The only symmetry operation for the P 1̅ space group is inversion through a point. Since this inversion is along a one-fold axis, it is equivalent to the centre of symmetry. Based on these symmetry operation, we can have two equipoint transformations (x,y,z and −x,−y,−z), yielding the general position multiplicity of 2 in the unit cell.

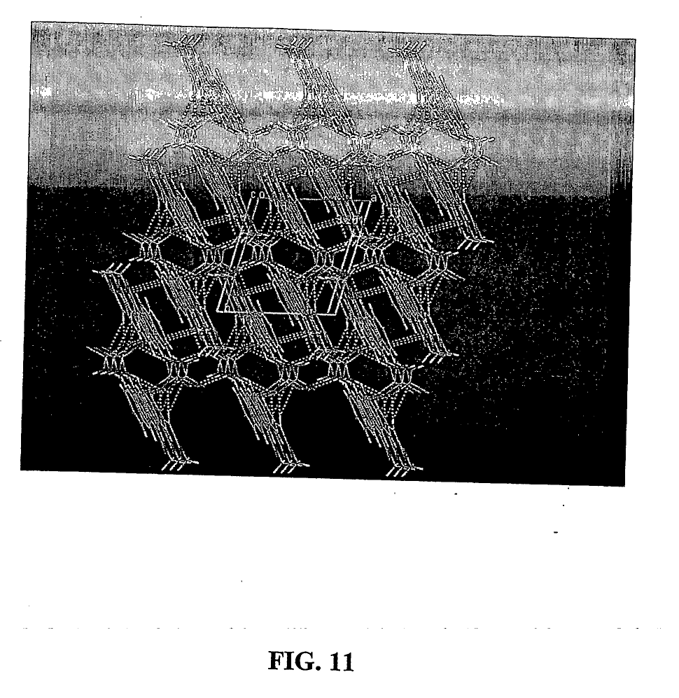

This salt adopts a specific molecular conformation, which promotes intermolecular hydrogen bonding. Like the hydrochloride salt, this drug molecule is monoprotonated, with a 1:1 molecular ratio between the drug molecule and the phosphate anion. In this structure, the three N—H donors (N(4), N(7), and N(14)) of the drug molecule and the oxygen acceptor of the phosphate anion participate in the hydrogen bonding. The oxygen O(12) and O(14) act as acceptors, and each forms two H-bond interactions, one with the protonated drug and the other with the phosphate anion. O(13) acts both as an acceptor (N(7)-H(10A) . . . O(13)) and a donor (O(13)-H(13B) . . . O(12)) to form H-bond interactions. O(11) acts as a donor, forming intermolecular H-bonds with the O(14) of the phosphate anion. The strong intermolecular N—H . . . O bond between the NHs of the cationic drug moiety and the oxygen of the anionic phosphate serve to link neighboring carbendazim molecules into chains. The O—H . . . O hydrogen bonds between the phosphate molecules allows the arrangement of the anion molecules into a line parallel to the b axis (FIG. 11).

TABLE 7
Geometrical parameters for hydrogen bonds observed in the
crystal lattice of carbendazim phosphate salt

| | d(H . . . A) | d(D . . . A) | ∠ (D-H . . . A) |
|---|---|---|---|
| D-H . . . A | (Å) | (Å) | (°) |
| N(7)—H(10A) . . . O(13) | 2.256 | 2.986 | 146.59 |
| N(14)—H(13A) . . . O(12) | 1.946 | 2.695 | 150.58 |
| N(5)—H(11A) . . . O(14) | 1.804 | 2.658 | 163.60 |
| N(7)—H(10A) . . . O(14) | 2.599 | 3.110 | 121.02 |
| O(13)—H(13B) . . . O(12) | 1.804 | 2.583 | 177.66 |
| O(11)—H(11B) . . . O(14) | 1.893 | 2.616 | 173.62 |

D and A refer to donor and acceptor atoms, respectively.

The packing arrangement of phosphate salt shows that both carbendazim and phosphate anions are arranged in stacks of parallel molecules, while the molecules in adjacent stacks are arranged in an inverted fashion. Within the stacks, the molecules are all arranged in the same direction. Apart from the routine intermolecular hydrogen bonds (Table 7), the most intense inter-molecular interactions in carbendazim occur among adjacent stacks between the carbon of the carbonyl group and the X system of the benzene ring. The observed bond length (C(3)-C(10)) of 3.261 Å is less than the van der Waals value of 3.4 Å. The strong electron acceptor character of the carbonyl oxygen induces the formation of δ-complexes.

Carbendazim Sulfate

Carbendazim sulfate crystallizes in the monoclinic system, having centrosymmetric space group C2/c. In monoclinic system, there is a “unique” axis—the one which is perpendicular to the other two. This unique axis is normally chosen as the b-axis, and thus, β≧90°. The crystal pattern for the sulfate salt is centered, unlike the hydrochloride or the phosphate salt that have primitive patterns. In a centered pattern, the grouping of motifs at the center of the rectangular cell is identical with that at the corners. The symbol ‘C’ indicates that the lattice is face-centered or end-centered, with a second lattice point lying at the center of the C-face (which is defined by the a- and b-axes). In such systems, the cell volume is double that of the primitive cell. In C2/c the symmetry operation is a 2-fold rotation axis parallel to the b-axis, and a glide plane perpendicular to the b-axis. The symbol ‘c’ indicates that the direction of glide is parallel to the c-axis. A glide plane combines the operation of reflection with that of translation, and therefore occurs only in extended arrays.

In this space group, 2 equipoint transformations are generated by point symmetry, which is doubled (to 4) by centrosymmetry. Since this crystal system is one face-centered, the number is further doubled to 8, thereby yielding the general position multiplicity of 8 in the unit cell. These general positions are (x,y,z), (−x,−y,−z), (½+x,½+y,z), (½−x, ½−y, −z), (−x,y,½−z), (x,−y,½+z), (½−x,½+y,½−z), and (½+x,½−y,½+z).

In the smallest molecular unit, SO₄²⁻ sits on a 2-fold axis such that only half of it is unique. The molecular ratio between the drug molecule and the anion is 2:1. Therefore, the asymmetric unit consists of a single protonated carbendazim molecule, one water molecule, and a half sulfate anion.

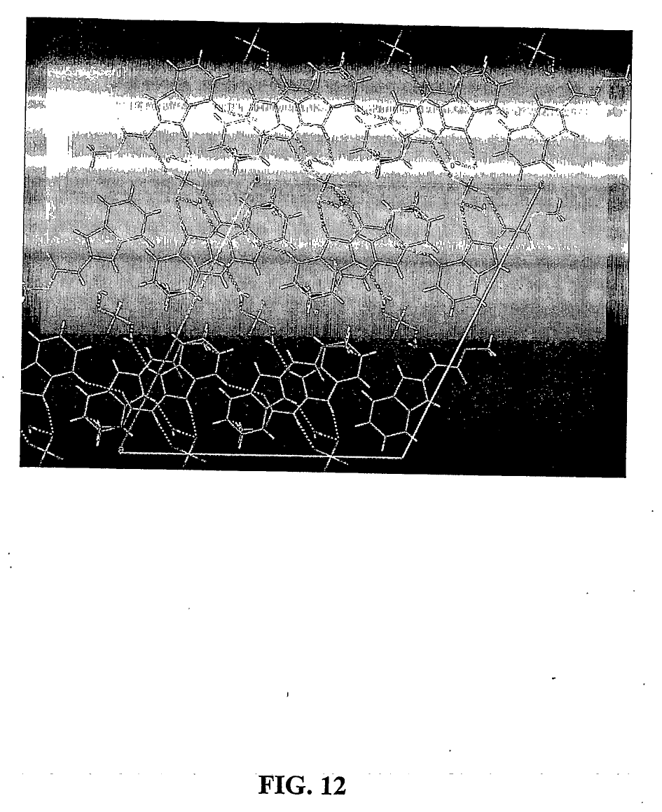

The sulfate salt adopts a molecular conformation which promotes intermolecular hydrogen bonding. In this structure, the three N—H donors of the drug molecule and the sulfate oxygen acceptor of the anion, along with the water molecule, participate in hydrogen bonding. Strong intermolecular hydrogen bonds between the NHs (both imidazole and carbamate) and the sulfate or water oxygens link the protonated carbendazim, the sulfate anions, and the water molecules into chains, which propogate along the b-axis (FIG. 12). Therefore, the packing diagram of this salt closely resembles that of the phosphate salt, having a column of anion, SO₄²⁻ in this case, running parallel to the b axis, where the sulfate molecules are hydrogen bonded to the water molecules (O(20)-H(21) . . . O(10) and O(20)-H(20) . . . O(11)). The drug molecule is again H-bonded to the anion on each side of the molecule, just as they do in the phosphate salt (N(7)-H(7A) . . . O(10), N(5)-H(5A) . . . O(1 1), N(5)-H(5A) . . . S(1) and N(7)-H(7A) . . . S(1). The drug molecule also forms hydrogen bonds with the water molecule (N(14)-H(14A) . . . O(20)).

TABLE 8
Geometrical parameters for hydrogen bonds observed in the
crystal lattice of carbendazim sulfate salt

| | d(H . . . A) | d(D . . . A) | ∠ (D-H . . . A) |
|---|---|---|---|
| D-H . . . A | (Å) | (Å) | (°) |
| N(7)—H(7A) . . . O(10) | 1.839 | 2.689 | 165.86 |
| N(5)—H(5A) . . . O(11) | 1.841 | 2.699 | 167.27 |
| N(14)—H(14A) . . . O(20) | 2.031 | 2.800 | 147.82 |
| N(5)—H(5A) . . . S(1) | 2.843 | 3.664 | 157.13 |
| N(7)—H(7A) . . . S(1) | 2.858 | 3.639 | 150.36 |
| O(20)—H(20) . . . O(11) | 2.082 | 2.900 | 160.92 |
| O(20)—H(21) . . . O(10) | 2.149 | 2.959 | 163.71 |
| C(9)—H(9A) . . . O(4) | 2.500 | 3.411 | 150.09 |

D and A refer to donor and acceptor atoms, respectively.

The packing arrangement of the sulfate salt shows the presence of intermolecular hydrogen bonds having carbon as the hydrogen donor. The C(9) of the benzene ring also acts as a hydrogen donor, forming a hydrogen bond with the oxygen of the carbonyl group (C(9) . . . O(4)=3.411 Å). Although it was realized as early as 1962 that an activated C—H group as present in some heterocyclic bases, for example, caffeine, theophylline, uric acid and related compounds, tend to interact with oxygen atoms in the same way as an O—H or N—H group and the short (<3.4 Å) C . . . O contacts observed in the crystals of these molecules were interpreted as C—H . . . O hydrogen bonds. It was not until 1982 that the existence of C—H . . . O hydrogen bonds in organic molecules was convincingly demonstrated and C—H . . . O bonds started gaining acceptance as a stabilizing force when adjusted within the framework of stronger forces, such as N—H . . . O, O—H . . . O hydrogen bonds and donor-acceptance interactions. Even though the C . . . O distance for the C—H . . . O bond in the sulfate salt is a bit higher than the limit, the linearity of the bond angle (150.09°) makes its existence more than possible.

Furthermore, the packing arrangement of the sulfate salt is stabilized by the presence of C—H . . . π interactions between the methyl group of one carbendazim and the benzene ring of the other. These interactions are C(11) . . . H(1C) (2.797 Å) and C(10) . . . H(1C) (2.676 Å).

Carbendazim Mesylate

The carbendazim mesylate salt, like the sulfate, crystallizes in a monoclinic system, although the mesylate salt has a different space group, Cc. Also, unlike other carbendazim sulfonate salts, the Bravais lattice for the mesylate salt is centered. The only symmetry operation associated with this space group is a glide plane in the direction parallel to the c-axis. This space group is chiral, having a z value of 4. The general positions are given by (x,y,z), (½+x,½−y,z), (x,−y,½+z), and (½+x,½−y,/½+z)).

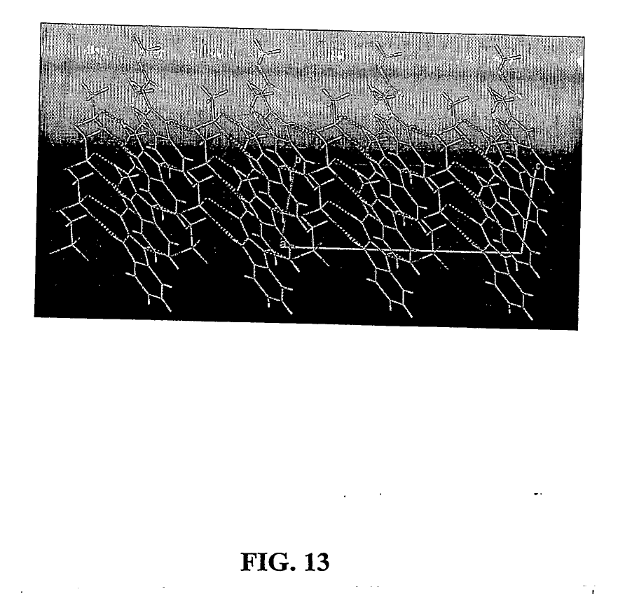

The asymmetric unit consists of one molecule each of protonated carbendazim and anionic methane sulfonate. A projection along the b-axis of the atomic arrangement of the salt is depicted in FIG. 13. The packing consists of alternate parallel stacks of protonated carbendazim and mesylate anions. These stacks are parallel to the a-axis (FIG. 13), and within each stack, all molecules are oriented in the same direction. These stacks of carbendazim and methanesulfonic acid are held together by intermolecular hydrogen bonds and C—H . . . π interactions. All the NHs on the carbendazim molecule act as hydrogen donors, whereas Os and S on mesylate anion behave as hydrogen acceptors. Every mesylate anion forms three N—H . . . O bonds, two bonds with the carbendazim molecule on the right side, and one with the other carbendazim molecule on the left side (FIG. 13). The N(5) of carbendazim can also form H-bond with the S(18) of the mesylate anion, though the N(5)-H(5A) . . . O(17) bond is more linear than the N(5)-H(5A) . . . S(18). The hydrogen bonding parameters are listed in Table 9.

Apart from the H-bonds, which are strong, single point interactions with a very well-defined geometry, there are other weaker, less well-defined interactions that are also responsible for holding the molecule together. One such interaction is C—H . . . π, where a polarized C—H group interacts with the aromatic ring. The presence of an electron withdrawing sulfonate,group polarizes the methyl group of the mesylate, making it electron deficient, which then interacts with the electron rich benzene ring of the carbendazim by forming C—H . . . π interactions. The distance of 2.759 Å between C(10)-H(19C) is smaller than the summation of their van der Waal radii (2.9 Å), which proves the presence of such interactions. Therefore, the hydrogen bonds and the van der Waals contacts give rise to a three-dimensional construction of the structure and add to the stability.

TABLE 9
Geometrical parameters for hydrogen bonds observed in the
crystal lattice of carbendazim mesylate salt

| | d(H . . . A) | d(D . . . A) | ∠ (D-H . . . A) |
|---|---|---|---|
| D-H . . . A | (Å) | (Å) | (°) |
| N(7)—H(7A) . . . O(15) | 1.827 | 2.696 | 168.80 |
| N(14)—H(14A) . . . O(16) | 1.934 | 2.745 | 152.59 |
| N(5)—H(5A) . . . O(17) | 1.850 | 2.730 | 178.77 |
| N(5)—H(5A) . . . S(18) | 2.901 | 3.713 | 154.10 |

D and A refer to donor and acceptor atoms, respectively.

Carbendazim Besylate

The carbendazim besylate salt, like the phosphate salt, crystallizes in the triclinic system and has a space group P 1̅, with Z=2. The asymmetric unit consists of one molecule each of carbendazim and benzenesulfonic acid. Carbendazim appears as a planar molecule in the asymmetric unit, with the benzenesulfonic acid perpendicular to it.

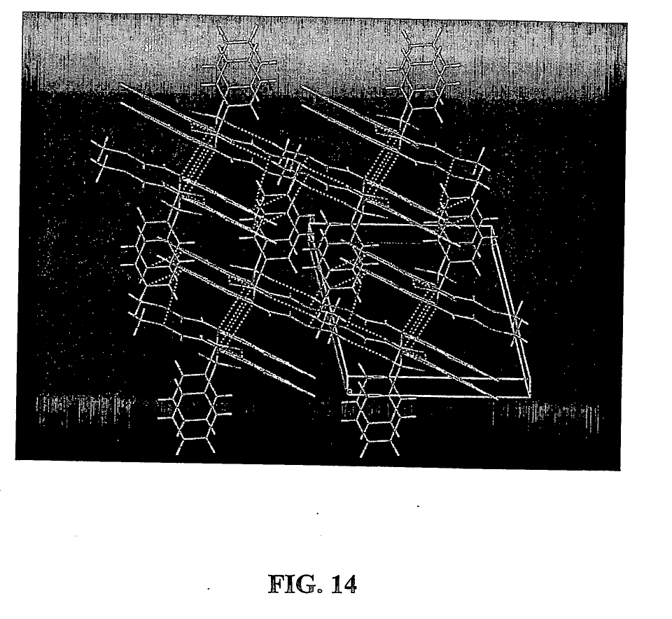

The packing arrangement of the salt shows the presence of intermolecular hydrogen bonding between the protonated carbendazim and the benzenesulfonate anion (FIG. 14). However, no intramolecular hydrogen bonding or intermolecular hydrogen bonding was observed between two carbendazim molecules or two benzenesulfonic acid molecules. As with the other salts, all hydrogen donating atoms (N) form hydrogen bonds with hydrogen acceptor atoms (O and S). Unlike the hydrochloride and sulfate salts, however, the packing motif of the besylate salt allows formation of two intermolecular hydrogen bonds involving carbon as the donor. The phenyl carbons (C(22) and C(23)) of the benzenesulfonate anion forms hydrogen bonds with the methoxy oxygen (O(2)) of the carbendazim molecule. The bond lengths (C . . . O) for both C—H . . . O bonds is less than 3.4 Å, and the bond angle is greater than 130°. Because of the intermolecular hydrogen bonds between carbendazim and benzenesulfonate anion, the carbendazim molecules are arranged along the b-axis.

TABLE 10
Geometrical parameters for hydrogen bonds observed in the
crystal lattice of carbendazim besylate salt

| | d(H . . . A) | d(D . . . A) | ∠ (D-H . . . A) |
|---|---|---|---|
| D-H . . . A | (Å) | (Å) | (°) |
| N(7)—H(7A) . . . O(17) | 1.916 | 2.789 | 171.48 |
| N(5)—H(5A) . . . O(16) | 1.908 | 2.772 | 166.76 |
| N(14)—H(14A) . . . O(15) | 2.011 | 2.797 | 148.10 |
| N(7)—H(7A) . . . S(18) | 2.894 | 3.686 | 150.67 |
| C(22)—H(22A) . . . O(2) | 2.574 | 3.302 | 133.68 |
| C(23)—H(23A) . . . O(2) | 2.588 | 3.334 | 135.67 |

D and A refer to donor and acceptor atoms, respectively.

From the packing arrangement of the besylate salt, it is clear that the stacks of carbendazim molecules are arranged perpendicular to the stacks of benzenesulfonate anions. Such arrangement is favorable for having T-shaped edge-to-face electrostatic interaction. Although benzene has no net dipole, it has an uneven distribution of charge, with greater electron-density on the face of the ring, and reduced electron-density on the edge, thus giving rise to quadrupole moments. Such quadrupole moments of the aromatic rings are thought be the precursors for the electrostatic component of the interaction. The possible edge-to-face interactions observed in besylate salt are H(9A) . . . C(23) and H(9A) . . . C(22), which have bond lengths of 2.696 Å and 2.736 Å, respectively.

Carbendazim Tosylate

The carbendazim tosylate salt crystallizes in the orthorhombic system having space group P2₁2₁2₁, which is chiral. This space group does not have any symmetry operations associated with inversion or mirror, and thus is devoid of a centre of symmetry and aptly defined as non-centrosymmetric. The symmetry operation for this space group involves both rotation and translation along a screw axis. In this space group, three two-fold screw axes are present along the a, b, and c directions. Thus, 2₁2₁2₁ mean that the asymmetric unit moves ½ of a repeat unit along the three axes for each ½ of a revolution about that axis. Because of these symmetry operations, 4 equipoint transformations are generated (x,y,z; ½+x, ½−y, −z; −x, ½+y, ½−z; ½−x, −y, ½+z), thereby having a general position multiplicity of 4 in the unit cell.

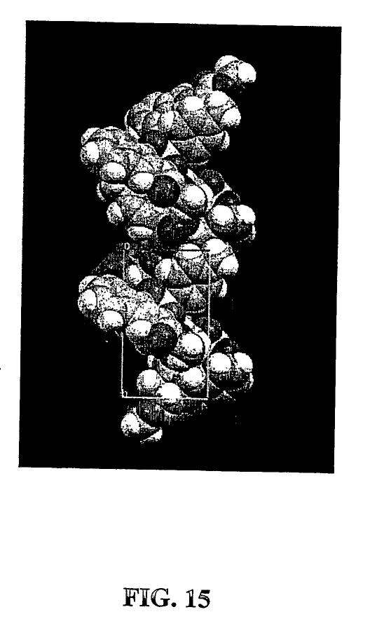

The asymmetric unit consists of one molecule each of carbendazim and toluenesulfonic acid. As seen in FIG. 9(f), the carbendazim molecule and the toluenesulfonic acid lie perpendicular to each other. The unit cell contains four molecules each of carbendazim and toluenesulfonic acid. The molecules are arranged in alternate layers of carbendazim and tosylate, in all three directions (FIG. 15). The carbendazim and tosylate molecules in the tosylate salt are arranged in infinite helices around a two-fold screw axis. When looking down the crystallographic c-axis, the carbendazim molecules within the stacks are flipped by 180°, whereas the tosylate molecules are oriented in the same direction. The molecules of adjacent stacks of carbendazim show an inclination angle of ±41.11° to the stack axis, which is the case a-axis in this case.

As expected, the packing arrangement of tosylate (FIG. 15) shows intermolecular hydrogen bonding (Table 11) between the NHs of carbendazim and Os and S of tosylate. The packing arrangement for tosylate, like besylate, shows a number of edge-to-face as well as CH-π interactions. These interactions are not only between the phenyl rings of carbendazim and p-toluenesulfonic acid but also between the methyl group of carbendazim and the phenyl ring of p-toluenesulfonic acid. Some of the noted interactions are H(9A) . . . C(23) (2.769 Å), H(9A) . . . C(24) (2.786 Å) and H(1A) . . . C(23)(2.779 Å).

TABLE 11
Geometrical parameters for hydrogen bonds observed in the
crystal lattice of carbendazim tosylate salt

| | d(H . . . A) | d(D . . . A) | ∠ (D-H . . . A) |
|---|---|---|---|
| D-H . . . A | (Å) | (Å) | (°) |
| N(7)—H(7A) . . . O(15) | 1.845 | 2.713 | 167.93 |
| N(5)—H(5A) . . . O(16) | 1.912 | 2.786 | 171.85 |
| N(14)—H(14A) . . . O(17) | 2.075 | 2.812 | 140.74 |
| N(7)—H(7A) . . . S(18) | 2.871 | 3.665 | 150.89 |
| N(5)—H(5A) . . . S(18) | 2.979 | 3.798 | 151.79 |
| C(25)—H(25B) . . . O(2) | 2.582 | 3.442 | 146.60 |

D and A refer to donor and acceptor atoms, respectively.

Packing Efficiency

Packing forces and crystal symmetry determine the chemical and physical properties of crystalline materials. The primary packing rule for molecular crystals, called Kitraigorodskii's Principle of Close Packing, is to maximize density and minimize free volume. Although void space between crystals is undesirable, it is usually unavoidable. The denser or more closely packed a crystal is, the lower the free energy will be, resulting in greater stability.

The packing efficiency can be interpreted by measuring the packing coefficient, κ, for a given crystal. The packing coefficient represents the amount of space filled by the molecules in a lattice, and is calculated as

$$K = N \frac{{V}_{vdw}}{{V}_{cell}}$$

where N is the number of molecules in the unit cell, v_{vdw} is the van der Waals volume of the molecule in the asymmetric unit, and v_{cell} is the volume of the unit cell. The van der Waals volumes presented here were calculated using the Conolly surface feature of the DS ViewerPro program and the standard van der Waals radii that are included in the software. The packing coefficients of various carbendazim salts are listed in Table 12. The packing coefficients for all the salts are between 0.65 and 0.73, which is in agreement with the ê range of 0.65-0.8 for stable crystals.

TABLE 12
Packing Coefficients of Carbendazim Salts

| Salt | N | V_{vdw}(Å) | V_{vdw}(Å) | κ |
|---|---|---|---|---|
| Hydrochloride | 4 | 206.438 | 1175.8 | 0.702 |
| Phosphate | 2 | 201.777 | 585.36 | 0.689 |
| Sulfate | 8 | 200.254 | 2198.7 | 0.728 |
| Mesylate | 4 | 207.539 | 1293.9 | 0.642 |
| Besylate | 2 | 257.771 | 765.01 | 0.674 |
| Tosylate | 4 | 273.072 | 1638.8 | 0.667 |

Moisture Sorption Studies

A high degree of moisture sorption or desorption by the salts at 30-50% RH, the expected humidity conditions of pharmaceutical manufacturing plants may create numerous handling and manufacturing difficulties including change in the drug's potency and true density, variation in flow properties, dissolution rates and bioavailability, as well as chemical instability. Although general trends have been noted between the propensities of salts to form hydrates and various structural features such as counter-ion radius and charge, a given salt may form several stoichiometric hydrates depending upon the crystallization conditions. Hence, an assessment of a compound's ability to adsorb moisture is an important developability criterion.

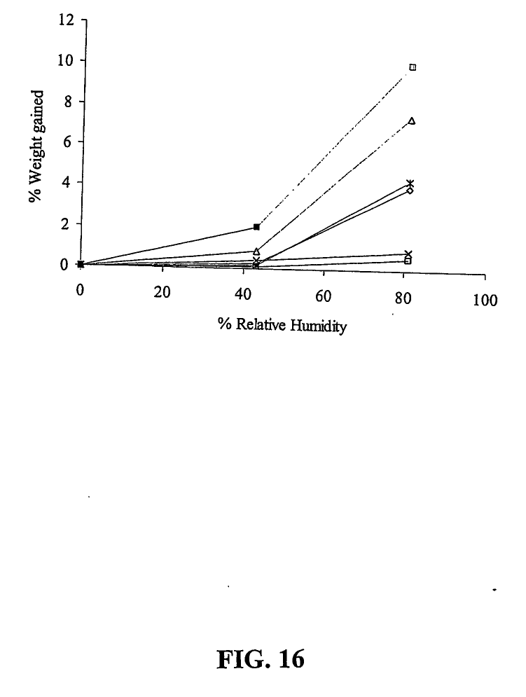

FIG. 16 shows the moisture adsorption curves for carbendazim salts under relative humidity values of 43 and 81%. As can be seen, the hydrochloride and sulfate salt were minimally hygroscopic, adsorbing less than 1% moisture at both 43 and 81% RH. Incidentally, both of these salts were synthesized as hydrates. In contrast, the phosphate and mesylate salts were found to be highly hygroscopic, adsorbing 7.5 and 10.1% moisture at 81% RH compared to 1-2% at 43% RH. The besylate and tosylate salts adsorbed less than 1% moisture at 43% RH, and approximately 4.3% at 81% RH.

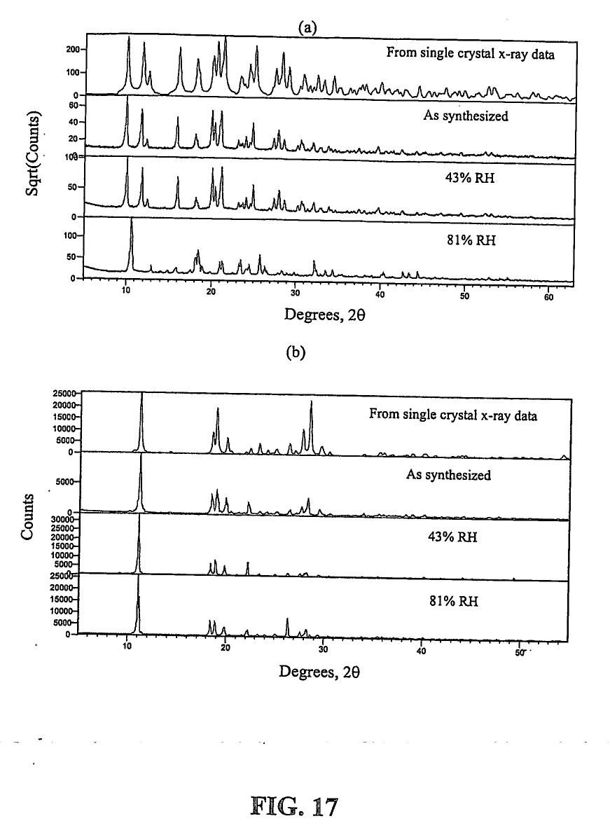

The powder x-ray diffraction patterns of hydrochloride, sulfate, besylate, and tosylate salts remained unchanged for the humidity values of 43 and 81%. However, both the mesylate and phosphate salts showed changes in their PXRD patterns for samples stored at 81% RH, which could be attributed to a change in the crystal form (FIGS. 17(a) and (b)). The mesylate sample stored at 81% RH lost intensity in the higher angle peaks as well as the major reflections at 11.7° 2θ and 20.3° 2θ, and gains a new peak at 18.8° 2θ, whereas the sample stored at 43% RH has similar PXRD pattern as the synthesized salt. The phosphate sample stored at 43% RH loses intensity in 28 0 2E reflection, while the 81% RH pattern has new reflection at 26° 2θ, and loses intensity in 22° 2θ.

These results indicate that at RH value of 43%, all salts adsorbed less than 2% moisture from the atmosphere. Although this caused the phosphate salt to change form, the other salts remained unchanged in their solid state form.

Solubility Studies

The aqueous solubilities of the hydrochloride, phosphate, sulfate, besylate, and tosylate salts of carbendazim at 25° C. are listed in Table 13. The mesylate salt was found to be highly soluble (>200 mg/ml), and therefore its saturable solubility was not determined. The solubility of sulfate reached a plateau at approximately 1.2×10⁻² M in the region below pH 1.58, indicating that the solution is saturated with respect to sulfate salt below this pH. On the other hand, the solubility of both besylate and the tosylate decreased below a pH value of 1.65 and 1.82, respectively. The solubility products of the different salts are also tabulated in Table 13.

TABLE 13
Solubility of different carbendazim salts

| | Water | | | | |
|---|---|---|---|---|---|
| | | S | | | ΔHₛ |
| Salt | pH | (mg/ml) | PHₘₐₓ | Kₛₚ (M²)^{b} | (J · K⁻¹ · M⁻¹) |
| Free Base | | 0.006 | NA | NA | 145.250 |
| Hydrochloride | 1.68 | 6.080 | NA | NA | 14.951 |
| Phosphate | 1.93 | 3.032 | 1.93 | 2.1 × 10⁻⁴ | 11.906 |
| Sulfate | 1.9 | 6.505 | 1.58 | 2.7 × 10⁻⁴ | 22.898 |
| Mesylate^{a} | 0.8 | 205.662 | NA | NA | NA |
| Besylate | 1.76 | 6.992 | 1.65 | 1.1 × 10−3 | 17.390 |
| Tosylate | 1.93 | 4.815 | 1.82 | 4.1 × 10⁻⁴ | 18.704 |

^{a}Saturation was not reached.
^{b}Ksp calculated after correcting for common ion effect.

The solubility temperature dependencies of the different salts were also studied. This is a semilogarithmic plot of solubility against reciprocal temperature. The slopes of such plots (or the tangent to a given curve at a given temperature, in the case of a nonlinear plot) yield the differential heat of solutions of the respective species. Usually such curves are nonlinear and ΔHₛ values are obtained calorimetrically at a given temperature. Within the limited temperature span of this investigation, the plots appear linear and rough estimates of ΔHₛ are possible. Table 13 lists ΔHₛ for all the studied compounds.

Dissolution Studies

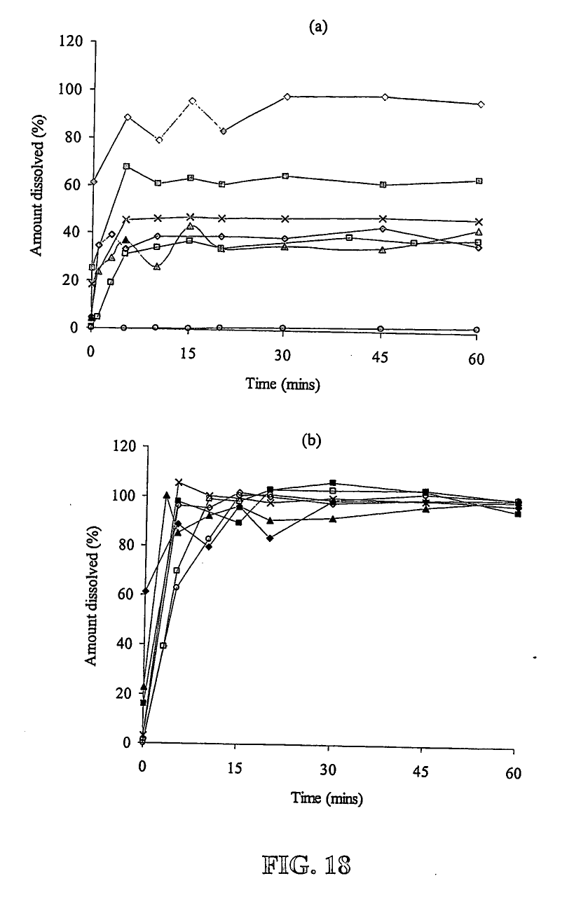

The dissolution behavior of the free base, as well as the hydrochloride, phosphate, sulfate, mesylate, besylate, and tosylate salts, were compared in Millipore water and 0.1N hydrochloric acid solution at pH 1.1. The pH 1.1 solution simulates the gastric fluid (pH 1-3 in the stomach), since the behavior in this solution is relevant to the bioavailability after oral administration. FIGS. 18a and b show the dissolution profiles in water and 0.1N HCl. As seen from FIG. 18(a) the hydrochloride, sulfate, besylate, tosylate, and phosphate salts as well as the free base form did not completely dissolve in water. Each of these five salts dissolved greater than 40% of the initial dose after 60 min. At the extremes, the free base dissolved less than 1% after 60 min, while the mesylate salt was completely soluble in water within just 30 min.

The dissolution of each salt in 0.1N HCl is shown in FIG. 18(b). The six salts and the free base were all completely soluble in this dissolution media. The time for dissolving 100% of sample crystals was different. The mesylate salt was instanteously soluble, whereas the sulfate and phosphate salt took 5-10 min to attain complete miscibility. The free base along with the hydrochloride, besylate, and tosylate dissolved completely in 15-20 min.

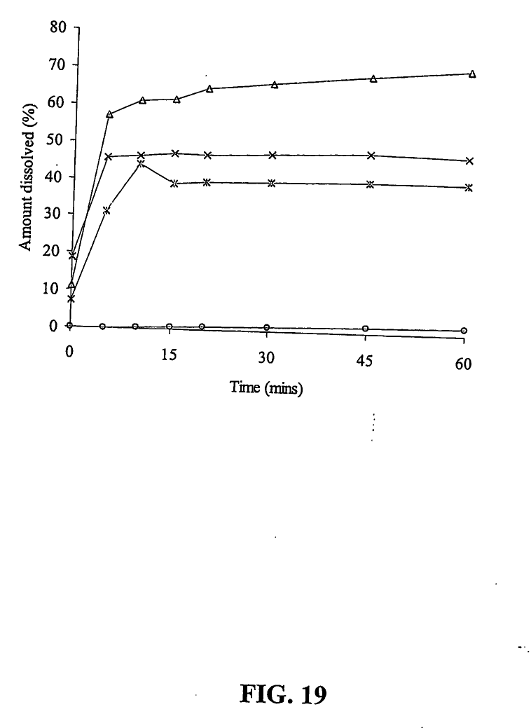

In order to compare the dissolution of prepared salts with physical mixtures of the free base and acid, carbendazim: phosphoric acid mixtures were prepared in the molar ratios of 1:1 and 1:2. FIG. 19 shows the dissolution profile of the two physical mixtures and the phosphate salt in Millipore water. The phosphate salt (found to be 1:1) shows better dissolution than the 1:1 physical mixture. On the other hand, the 1:2 physical mixture shows better dissolution than the phosphate salt. Due to excessive acid, it is believed the 1:2 physical mixture decreases the pH of the diffusion layer in the microenvironment of the particles more than the 1:1 phosphate salt, thereby facilitating dissolution.

A salt exhibits a higher dissolution rate than the base at any given pH, despite having the same equilibrium solubilities. It is believed the salt effectively acts as its own “buffer” to alter the pH of the diffusion boundary layer, thereby increasing the apparent solubility of the parent drug within that layer. Thus, administration of basic drugs in their salt forms ensures that stomach emptying, rather than in vivo dissolution, will be the rate-limiting factor in its absorption. From the dissolution studies it is evident that the formed salts have better dissolution than the free base. In water, mesylate dissolved the fastest. In 0.1N hydrochloric acid, the given amount of all six salts (equivalent to 50 mg carbendazim) as well as the free base (50 mg) dissolved completely within 20 min. The phosphate salt (1:1) had better dissolution than the 1:1 physical mixture of carbendazim and phosphoric acid, while the 1:2 physical mixture had the greatest dissolution of the three due to the excess amount of phosphoric acid.

A number of acidic salts of the weak ampholyte carbendazim were synthesized in order to increase the apparent solubility. A preformulation study was conducted on all the synthesized salts. Table 14 lists the physical properties of the studied salts along with the free base. All of the salts showed better dissolution rate profiles than the free base, with mesylate having the best dissolution time. The hydrochloride and the sulfate salts were synthesized as hydrates, and were found to exist in more than one form.

TABLE 14
Comparison of some basic properties of carbendazim and six salts

| Property | Base | Hydrochloride | Phosphate | Sulfate | Mesylate | Besylate | Tosylate |
|---|---|---|---|---|---|---|---|
| Appearance | Light grey, | White, | White, | White, | White, | White, | White, |
| | crystalline | crystalline | crystalline | crystalline | crystalline | crystalline | crystalline |
| Crystal System | NA | Orthorhombic | Triclinic | Monoclinic | Monoclinic | Triclinic | Orthorhombic |
| Space group | NA | P2₁2₁2₁ | P1 | C2/c | Cc | P1 | P2₁2₁2₁ |
| Mol. Wt. | 191.2 | 263.68 | 289.2 | 516.49 | 287.29 | 363.39 | 349.36 |
| MP (° C.) | 240 | 118.76 | 189.54 | 137.31 | 202.45 | 211.23 | 216.47 |
| Hydrate | anhydrous | dihydrate | anhydrous | monohydrate | anhydrous | anhydrous | anhydrous |
| Polymorphism | No evidence of | Atleast three | No evidence of | Atleast two | No evidence of | No evidence of | No evidence of |
| | polymorphs | forms detected | polymorphs | forms detected | polymorphs | polymorphs | polymorphs |
| S_{w} (mg/ml) | 0.006 | 6.080 | 3.032 | 6.505 | >205.662* | 6.992 | 4.815 |
| pH of saturated | 5.90 | 1.68 | 1.93 | 1.90 | 0.80 | 1.76 | 1.93 |
| sol. | | | | | | | |
| t_{60%} (min) pH 1 | 5 | 2-3 | 2-3 | 2-3 | 1 | 2-3 | 2-3 |
| t_{40%} (min) water | NA | ˜5 | ˜5 | ˜3 | <1 | ˜5 | ˜5 |
| Hygroscopicity | Gain 0.36% | Gain 0.13% | Gain 0.88% | Gain 0.38% | Gain 2.04% | Gain 0.18% | Gain 0.27% |
| (hygrostat for 8 | moisture | moisture | moisture, | moisture | moisture, same | moisture | moisture |
| days at 43% RH) | | | PXRD pattern | | PXRD pattern. | | |
| | | | changed. | | | | |

*solution not saturated with mesylate salt
t_{40%} and t_{60%} represents time for dissolution of 40% and 60% of given amount of salt/free base (equivalent to 50 mg carbendazim)

Acid Formulations

Free acid in formulations comprising salts of weak base compounds described herein results in improved dissolution of the weak base compound. For example, the dissolution can be faster or more complete than in formulations not containing additional acid. The ratio of salt of weal( base to free acid can be any ratio, however about 1:0.5 to about 1:3 are particular useful ratios, including all intermediate values and ratios therein. Particular examples of compositions include a phosphoric acid salt of a weak base combined with phosphoric acid free acid in the ratios described above. Other particular examples of compositions of the invention include a chloride salt of a weak base combined with a hydrochloric acid free acid in the ratios described above. Other particular examples of compositions of the invention include a sulfate salt of a weak base combined with a sulfuric acid free acid in the ratios described above. Other particular examples of compositions of the invention include a mesylate salt of a weak base combined with a methanesulfonic acid free acid in the ratios described above. Other particular examples of compositions of the invention include a besylate salt of a weak base combined with a benzenesulfonic acid free acid in the ratios described above. Other particular examples of compositions of the invention include a tosylate salt of a weak base combined with a toluenesulfonic acid free acid in the ratios described above. The free acid can be the same as the acid used to prepare the salt, or can be different. The free acid can also be a mixture of the acid used to prepare the salt and one or more acids not used to prepare the salt. One particular weak base useful in formulations is carbendazim.

In order to evaluate the importance of free acid in formulations, dissolution profiles of carbendazim tosylate salt and an equimolar physical mixture of carbendazim tosylate salt—p-toluenesulfonic acid were compared. The dissolution study was done in Millipore treated water. A weighed amount of salt or physical mixture (having equivalent of 50 mg free carbendazim) was triturated so as to have uniform particle size and suspended in 250 ml Millipore treated water. The stirring rate was maintained at 250 rpm and the study was performed at room temperature. 1 ml aliquot samples filtered through 0.45 μm Millipore filter was withdrawn as 1, 5, 10, 15, 20, 30, 45, and 60 min, respectively. 1 ml of the dissolution media was added to the dissolution vessel after each sampling period to maintain constant volume. The samples were analyzed by a HPLC procedure.

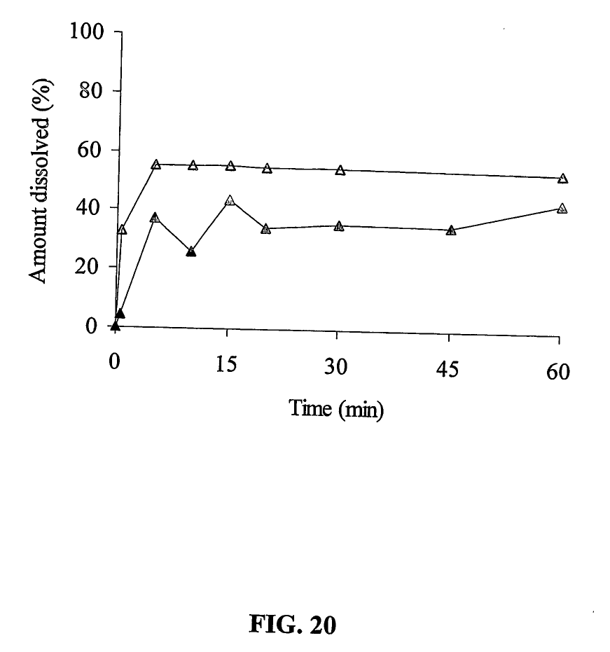

The dissolution of carbendazim tosylate salt and the physical mixture of carbendazim tosylate salt—p-toluenesulfonic acid are shown in FIG. 20. It is clear that the physical mixture shows better dissolution than the salt alone.

Although the description herein contains many specificities, these should not be construed as limiting the scope of the invention, but as merely providing illustrations of some embodiments of the invention. For example, weak base salts other than those specifically described herein can be made using the description provided herein. All references cited herein are hereby incorporated by referenced to the extent not inconsistent with the disclosure herewith.

We claim:

1. A pharmaceutical composition comprising a salt of a weak base compound of formula: 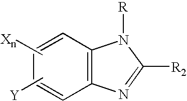
wherein X is hydrogen, halogen, alkyl of less than 7 carbon atoms or alkoxy of less than 7 carbon atoms; n is a positive integer of less than 4; Y is hydrogen, chlorine, nitro, methyl, ethyl or oxychloro; R is hydrogen, alkylaminocarbonyl wherein the alkyl group has from 3 to 6 carbon atoms or an alkyl group having from I to 8 carbons and R₂ is 4-thiazolyl, NHCOOR₁ wherein R₁ is aliphatic hydrocarbon of less than 7 carbon atoms, or an alkyl group of less than 7 carbon atoms;
one or more free acids; and
optional pharmaceutical additives, wherein the salt and one or more free acids are present in the composition at a ratio of 1:0.5 to 1:3 by weight.

2. The pharmaceutical composition of claim 1, wherein the salt is one or more selected from the group consisting of: chlorides, bromides, phosphates, sulfates, tosylates, benzoylates, nitrates, sulfonates, formates, tartrates, maleates, malates, citrates, benzoates, salicylates, ascorbates and mesylates.

3. The pharmaceutical composition of claim 2, wherein the salt is one or more selected from the group consisting of: chlorides, phosphates, sulfates, tosylates, benzoylates and mesylates.

4. The pharmaceutical composition of claim 1, wherein the salt and the free acid are present in the composition in a weight ratio of 1:1.

5. The pharmaceutical composition of claim 1, wherein the salt and the free acid are present in the composition in a weight ratio of 1:2.

6. The pharmaceutical composition of claim 1, wherein the salt is crystalline.

7. The pharmaceutical composition of claim 1, wherein the pH of an aqueous solution or suspension of the composition is 2 or less.

8. The pharmaceutical composition of claim 1, wherein the weak base compound is an imidazole derivative.

9. The pharmaceutical composition of claim 8, wherein the weak base compound is 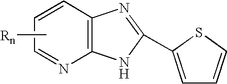
where n is an integer from 1 to 3 and R is hydrogen, alkyl having from 1 to 7 carbon atoms, chloro, bromo, fluoro, oxychloro, hydroxy, sulfhydryl or alkoxy having the formula —O(CH₂)_{y}CH₃ wherein y is an integer from 0 to 6.

10. The pharmaceutical composition of claim 1, wherein the weak base compound is a benzimidazole derivative.

11. The pharmaceutical composition of claim 10, wherein the weak base compound is carbendazim.

12. The pharmaceutical composition of claim 1, wherein the weak base compound is a pyridine derivative.

13. The pharmaceutical composition of claim 1, wherein the weak base compound is an aniline derivative.

14. The pharmaceutical composition of claim 1, wherein the composition is used for oral, intravenous or infusion administration.

15. The pharmaceutical composition of claim 1, wherein the free acid has the same anion as the salt.

16. The pharmaceutical composition of claim 15, further comprising a free acid having a different anion as the salt.

17. The pharmaceutical composition of claim 1, wherein the free acid has a different anion as the salt.

18. A solution or suspension of the pharmaceutical composition of claim 1.

19. A crystalline salt of a weak base compound of formula: 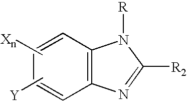
wherein X is hydrogen, halogen, alkyl of less than 7 carbon atoms or alkoxy of less than 7 carbon atoms; n is a positive integer of less than 4; Y is hydrogen, chlorine, nitro, methyl, ethyl or oxychloro; R is hydrogen, alkylaminocarbonyl wherein the alkyl group has from 3 to 6 carbon atoms or an alkyl group having from 1 to 8 carbons, and R₂ is 4-thiazolyl, NHCOOR₁ wherein R₁ is an aliphatic hydrocarbon of less than 7 carbon atoms, or an alkyl group of less than 7 carbon atoms;
wherein the salt is selected from the group consisting of: hydrochloride, phosphate, sulfate, tosylate, benzoylate and mesylate.

20. The crystalline salt of claim 19, further comprising one or more free acids.

21. A method of treating disease, comprising administering to a patient a pharmaceutically active amount of a pharmaceutical composition of claim 1.
